(19) United States
(12) Patent Application Publication
(10) Pub. No.: US 2023/0097883 A1
(43) Pub. Date: Mar. 30, 2023

(54) ERGOTHIONEINE PET COMPOUNDS AND METHODS OF USE THEREOF

(71) Applicant: VANDERBILT UNIVERSITY, Nashville, TN (US)

(72) Inventors: Wellington PHAM, Nashville, TN (US); William J. Behof, Nashville, TN (US); Clayton Whitmore, Nashville, TN (US); Dmitry Koktysh, Nashville, TN (US); Justin Haynes, Nashville, TN (US)

(21) Appl. No.: 17/823,707

(22) Filed: Aug. 31, 2022

Related U.S. Application Data
(60) Provisional application No. 63/239,875, filed on Sep. 1, 2021.

(51) Int. Cl. A61K 51/04

(52) U.S. Cl. CPC A61K 51/0453

(57) ABSTRACT

The present disclosure includes precursor compounds and probes such as ergothioneine (ERGO) based positron emission tomography (PET) (ERGO PET) probes or compounds or compositions including the ERGO PET probe such as pharmaceutical compositions, methods of making the ERGO PET probes or compositions, methods of imaging using the ERGO PET probes or compositions, and the like. The present disclosure provides for ERGO PET probes or compositions that can be used for imaging, diagnosing, localizing, monitoring, and/or assessing neurodegenerative diseases and inflammatory diseases or related biological conditions.

### CROSS-REFERENCE TO RELATED APPLICATIONS

This application claims the benefit of and priority to U.S. Provisional Application Ser. No. 63/239,875, having the title “ERGOTHIONEINE PET COMPOUNDS AND METHODS OF USE THEREOF” filed Sep. 1, 2021, the disclosure of which is incorporated herein in by reference in its entirety.

### FEDERAL FUNDING

This invention was made with government support under grant number R01AG061138 awarded by the National Institutes of Health (NIH). The government has certain rights in the invention.

### BACKGROUND

L-ergothioneine (ERGO) is a food-derived hydrophilic antioxidant available in fungi and various bacteria, but not in animals or higher plants. It has been known as an antioxidant since its discovery a century ago from fungi of the genus Claviceps purpurea. Chemically, this rare betaine-based amino acid has a similar chemical structure to histidine but with the presence of a sulfhydryl moiety on the imidazole ring. The molecule exists as a tautomeric form between thioketone and thiol derivatives (FIG. 1), albeit the former predominates at physiological pH. Because plants and animals cannot produce ERGO, it must be obtained from the diet. Mushrooms, in particular are a rich source of ERGO. As an antioxidant agent ERGO is capable of preventing cell and tissue damage, a key contributor to aging, by protecting against free radicals and oxidative stress. Its acquired adaptive antioxidant capability for the protection of injured tissues could be the reason for the observations that the highest concentration of ERGO are usually found in the red blood cells of old-age individuals, brain, liver, kidney, ocular tissues, and injured tissues. Additional evidence suggests that ERGO could target mitochondria and dampen the excess of mitochondria-specific ROS in response to oxidative stress.

ERGO is also implicated in a number of neurological pathways. Substantial research data indicate that ERGO is a physiological antioxidant cytoprotectant. Protection against cytotoxicity elicited by Cu (II), hydrogen peroxide, iron, and sodium nitrite is derived from the conspicuous affinity of ERGO for metal cations, such as Fe and Cu, permitting capture and neutralization of associated radicals. It has been demonstrated that ERGO concentration decreases significantly with age, and markedly lower levels were found in individuals with mild cognitive impairment compared to the controls, supporting the potential for ERGO deficiency to act as a risk factor for neurodegeneration. It is worthwhile to mention that ERGO does not permeate the blood-brain barrier (BBB); its uptake in cells is mediated by an OCTN1 (organic cation transporter novel, type 1) receptor. Other studies have shown that ERGO can protect neurons both in vitro and in vivo against a spectrum of stressors. Taken altogether, these data strongly suggest that ERGO is involved in healthy aging, serving as a “longevity vitamin”. The term “vitamin” is used in here since ERGO is a micronutrient, that the body does not need a lot of to function, but the material needs to be constantly available by uptake from an external source such as foods or supplements and it therefore does fit into that category.

To date, all preclinical studies had relied solely on in vitro analysis of ERGO using analytical and bioassay methods for analyzing the ERGO distribution from extracted tissues. The same holds true for clinical studies to validate the benefit of ERGO consumption in the diet; serum samples were collected for assessing the ERGO levels.

### SUMMARY

Embodiments of the present disclosure relates to ergothioneine PET compounds and methods of use thereof. In particular, the presently disclosed subject matter relates to ergothioneine PET analogs and imaging methods using these compounds.

The present disclosure provides for an ergothioneine (ERGO) based positron emission tomography (PET) probe having the following structure:

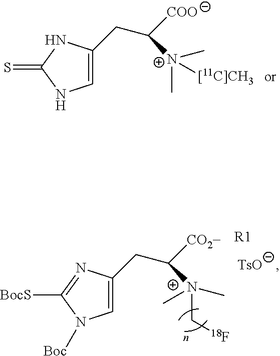

wherein n is 1, 2, or 3, wherein R1 is an alkyl group.

The present disclosure provides for a pharmaceutical composition, comprising: a pharmaceutical carrier and an effective dose of ergothioneine (ERGO) based positron emission tomography (PET), wherein the ERGO PET probe has the following structure:

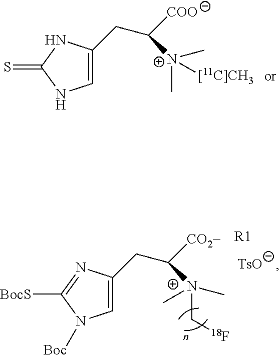

where n is 1, 2, or 3, where R1 is an alkyl group.

The present disclosure provides for a precursor compound for ergothioneine (ERGO) based positron emission tomography (PET) probe having the following structure:

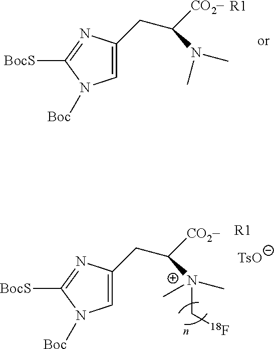

wherein n is 1, 2, or 3, wherein R1 is an alkyl group. The present disclosure also includes a kit comprising the precursor compound for ERGO PET probe

The present disclosure provides for a method of imaging an inflammatory disease in a subject comprising: administering to a subject a labeled probe in a detectably effective amount, wherein the labeled probe is ergothioneine (ERGO) based positron emission tomography (PET) probe having the following structure:

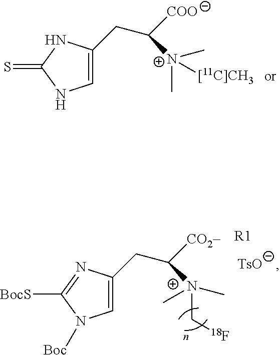

wherein n is 1, 2, or 3, wherein R1 is an alkyl group; imaging at least a portion of the subject; and detecting the labeled probe, wherein the location of the labeled probe corresponds to inflammation corresponding to the inflammatory disease.

The present disclosure provides for a composition, comprising:

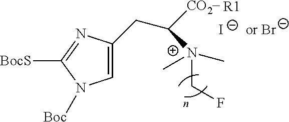

wherein n is 1, 2, or 3, wherein R1 is an alkyl group.

The present disclosure provides for a pharmaceutical composition, comprising: a pharmaceutical carrier and an effective dose of

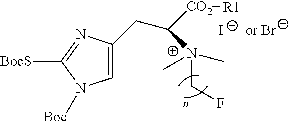

wherein n is 1, 2, or 3, wherein R1 is an alkyl group.

### BRIEF DESCRIPTION OF THE DRAWINGS

The presently disclosed subject matter will be better understood, and features, aspects and advantages other than those set forth above will become apparent when consideration is given to the following detailed description thereof. Such detailed description makes reference to the following drawings, wherein:

FIG. 1.1 shows a schematic illustrating tautomerized isoforms of ERGO at physiological pH.

FIG. 1.2 shows a schematic illustrating retrosynthetic analysis of a precursor for generating a [¹¹C]ERGO PET radioligand.

FIG. 1.3 shows schematics illustrating [¹¹C]ERGO PET radioligand synthesis and characterization.

FIG. 1.4 shows graphs illustrating chromatograms of [¹¹C]ERGO and “cold” ERGO.

FIGS. 1.5A-B show images and graphs illustrating activity in the brain of a mouse (n=4) injected with [¹¹C]ERGO simultaneously with the start of 90 min dynamic acquisition in a microPET. (1.5A) PET/CT image outlining the skull of the mouse at different timepoints. (1.5B) The time-activity-curves (TAC) within different regions of the brain. % ID/g: percentage of injected dose per animal weight.

FIGS. 1.6A-C show images and graphs illustrating oxidative stress in the LPS-induced neuroinflammation mouse model using [¹¹C]ERGO radioligand. (1.6A) Representative view of the PET signal (white arrows) depicted from the brain of LPS-treated (n=3) versus non-treated control mice (n=3) and LPS-treated mice, co-injected with Tempol (n=3). The immunohistochemical analysis of the hippocampal region using anti-GFAP antibodies. The observed fluorescent signal was indicated in red squares. (1.6B) Comparison of PET signal in different regions of the brain. (1.6C) Semi-quantitative analysis of the uptake in the subregions of the brains of LPS-treated versus control and LPS-treated+Tempol. *, P<0.05; **, P<0.005, #, P=0.05.

FIG. 1.7 shows a schematic and graphs illustrating ex-vivo cut-and-count biodistribution of I.V.-injected [¹¹C]ERGO radioligand in WT mice. At 5 (n=5), 10 (n=5) or 30 (n=6) min post I.V. injection, animals went through cardiac perfusion before tissues collections and counted. Tissue radioactivity was assessed and expressed as % ID/g.

FIG. 2.1 shows the design and synthesis of an ERGO precursor for [¹¹C]CH₃ labeling.

FIG. 2.2 shows radiolabeling to generate [¹¹C]ERGO radioligand. After labeling, the Boc groups were deprotected in TFA, while the methyl ester was cleaved in 5M NaOH at an elevated temperature. The RP-HPLC chromatogram of the co-injection data of the ERGO compound, which was detected in the UV channel (254 nm) along with [¹¹C]ERGO radioligand, detected in the radiometric channel. The radioactive compound [¹¹C]ERGO elutes at 7.6 min, while the “cold” compound elutes at 7.3 min.

FIG. 2.3 illustrates a set of representative data of a 30-min dynamic microPET imaging of the distribution and retention of [¹¹C]ERGO radioligand with a focus on the brains of 5XFAD mice (2.3A, 2.3B, 2.3C, 2.3D) versus WT control (2.3E, 2.3F, 2.3G, 2.3H) (n=4, each). Each animal received 14.8 MBq of [¹¹C]ERGO in 0.1 mL via the lateral tail vein. The uptake was quantified and compared using ImageJ (2.3G). Data are shown as the mean±SEM for all brain subregions, p<0.05.

FIG. 2.4 illustrates representative fluorescence images of activated astrocytes and microglia using anti-GFAP and anti-IBA1 protein markers, respectively in 5XFAD versus WT mice. The coronal sections of the mouse hippocampus were stained with DAPI (nuclear staining, blue, 465 nm channel) to provide anatomical laminar landmarks. The GFAP expression (green, 517 nm channel, scale bar 100 μm) is prominent across the hippocampal regions of 5XFAD compared to those from WT counterparts. The GFAP signal was quantified, and the signal distribution was scored on an ordinal scale after thresholding using the Otsu method and presented in the bar graph. An asterisk indicates significant differences between WT vs. 5XFAD (*p<0.05). Meanwhile, IBA1 immunohistochemistry (red, 696 nm channel, scale bar 200 μm) reveals a significant increase in microglial in 5XFAD vs. WT mice (**p<0.01).

FIG. 2.5 illustrate a mechanism of acidic catalyzed hydrolysis of (A) a Boc group and (B) t-butyl ester.

FIG. 3.1A illustrate the chemical structures of ERGO and the tautomerized isoforms at physiological pH. FIG. 3.1B illustrates the hypothesis is that ROS and transition metals in oxidative stress environments could be involved in the initiation of Abeta aggregation; with the capability to scavenge both ROS and metals, ERGO could prevent this event. FIG. 3.1C illustrates the 2D-HPLC was equipped with a nickel column to demonstrate the strong affinity of ERGO for metals. Upon exposure to the nickel column, His tag-Abs would be immobilized onto the column due to the strong binding of His tag (6 histidines) for the metal. The His tag-Abs can be eluted from the column using a buffer with a high concentration of imidazole (left figure). However, in the presence of ERGO (14.5 mM), the His tag-Abs were washed out immediately (right figure). FIGS. 3.1D and 3.1E illustrate ERGO scavenges copper and iron, respectively. In these assays, Cu(II) or Fe(II) forms a complex with pyrocatechol violet (PV) dye or ferrozine, resulting in a chromophore with strong absorbance at the wavelengths of 632 nm (copper) or 562 nm (iron). However, in the presence of ERGO, these transition metals were scavenged, leading to decreased PV-metal and ferrozine-metal complex concentrations, which resulted in a loss of absorbance at respective wavelengths. FIGS. 3.1F and 3.1G illustrates that in this cell-based assay, Hela (3.1F) or neuroblatoma SH-SY5Y cells (3.1G) were treated with DCFH-DA dye, which will diffuse into the cytoplasm and be trapped there after deacetylation caused by cellular esterases. The resulting nonfluorescent dye was oxidized by free radicals emitting a robust fluorescent signal (red curve). ERGO scavenges free radicals in the cells and leads to fluorescence attenuation. The fluorescence quenching is concentration dependent. At a dose of 100 mM, ERGO effectively quenches the fluorescence totally. Each point is the average of a pentaplicate assay. p<0.0001.

FIG. 3.2 illustrates the timeline of the therapy. Three independent cohorts of animals were treated with ERGO ranging from 25-50 mg/Kg via gavage 3 times per week for 4 weeks with mice from each group represented in each cohort. Afterwards, the animals underwent behavioral testing and PET imaging to assess the molecular biomarkers related to AD. Finally, animals were sacrificed, and brain sections were prepared for immunohistochemical analysis. FIG. 3.2A illustrates a schematic of experimental time-line. FIG. 3.2B illustrate elevated zero maze, percent time spent in closed zones during 5-min trial. FIG. 3.2C illustrates Exploratory locomotor activity, total distance travelled across a 30-min trial. FIG. 3.2D illustrates rotarod, latency to fall across 3 days of testing, mean from 3 daily trials. FIG. 3.2E illustrates the novel object recognition, discrimination index (T_{Novel}/(T_{Novel}+T_{Familiar})). FIGS. 3.2F and 3.2G illustrates the conditioned fear task, FIG. 3.2F illustrates context-based recall, time spent freezing across 4-min trial. FIG. 3.2G illustrates cue-based recall, time spent freezing during “cue off” (2 minutes) and “cue-on” (2 minutes) portions of the trial. Wild-type control n=6 male, n=6 female, non-treated 5XFAD n=6 male, n=6 female, ERGO-treated 5XFAD n=9 male, n=9 female; (D) p<0.001, Main effect of training day; (G) * p<0.05, ***p<0.0001 Cue-on freezing time compared to cue-off freezing time for each group.

FIG. 3.3 illustrates a reduced Abeta expression in the brains of ERGO-treated 5XFAD mice. After the therapy, all cohorts of animals were screened non-invasively using PET/CT scans to assess the levels of Abeta using [¹¹C]PIB PET radioligand. The background PET signal was first established using WT mice (3.3A, 3.3B, 3.3C) (n=4). A similar process was performed on non-treated 5XFAD mice, and the data demonstrated significant uptake and retention of [¹¹C]PIB, reflecting the enhanced expression of Abeta (3.3D, 3.3E, 3.3F) (n=5). While PET signal was much lower, suggesting less Abeta loads in the treated 5XFAD mice (3.3G, 3.3H, 3.3I) (n=7). Semi-quantitative analysis of the in vivo uptake of [¹¹C]PIB PET radioligand in different subregions of the brains of three cohorts of mice (J); p<0.05 for each subgroup. The in vivo PET data corroborate with Abeta immunohistochemistry (green, 488 nm channel) as shown in representative staining of Abeta on DAPI (blue)-stained coronal brain slices (10-μm) using 6E10 antibodies of ERGO-treated versus non-treated 5XFAD mice (n=3, each cohort, 3 slides per mouse) and subsequent quantitative analysis of the pixel counts after thresholding for the hippocampus (3.3K, 3.3L, 3.3M), pyramidal cortex (3.3N, 3.3O, 3.3P) and thalamus (3.3Q, 3.3R, 3.3S). *p<0.05, ***p=0.0004.

FIG. 3.4 illustrates the attenuation of oxidative stress in ERGO-treated 5XFAD mice using the [¹¹C]ERGO radioligand. Representative of in vivo PET imaging data to assess oxidative stress of non-treated 5XFAD (n=3) (3.4A, 3.4B, 3.4C) versus ERGO-treated 5XFAD (n=5) (3.4D, 3.4E, 3.4F). The axial (3.4A, 3.4D), coronal (3.4B, 3.4E) and sagittal (3.4C, 3.4F) images were obtained from the PET/CT scans. Semi-quantitative analysis of the in vivo uptake of [¹¹C]ERGO PET radioligand in different subregions of the brains of two cohorts of 5XFAD mice (3.4G); the difference between treated and non-treated cohorts is quantified with p<0.05 for all subregions of the brain. Representative of immunohistochemical staining and the corresponding quantification data of GFAP-positive astrocytes (3.4H, 3.41, 3.4J) (green, 488 nm channel) and IBA1-positive activated microglia (3.4K, 3.4L, 3.4M) (green, 488 nm channel) on coronally DAPI stained (blue) brain slices (10 μm) of non-treated 5XFAD (3.4H, 3.4K) versus ERGO-treated counterpart (3.41, 3.4L) (n=5, each), *p<0.05, ***p=0.0007.

FIG. 3.5 illustrates the imaging of glucose metabolism using [¹⁸F]FDG PET radioligand. Representative of 20 min dynamic PET scans with focus on the brains of WT mice (3.5A, 3.5B, 3.5C), non-treated 5XFAD (3.5D, 3.5E, 3.5F) and ERGO-treated 5XFAD (3.5G, 3.5H, 3.51). The uptake of the [¹⁸F]FDG PET probe was quantified as standard uptake values (SUV) in the brains and the SUVs were compared between ERGO-treated and non-treated 5XFAD (n=3, each) at p<0.05 (3.5J).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

In general, the present disclosure relates to ergothioneine PET compounds and methods of use thereof. In particular, the presently disclosed subject matter relates to ergothioneine PET analogs and imaging methods using these compounds.

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of chemistry, medical imaging, biochemistry, pharmacology, medicine, and the like, which are within the skill of the art. Such techniques are explained fully in the literature.

Prior to describing the various embodiments, the following definitions are provided and should be used unless otherwise indicated.

Unless otherwise defined, all technical and scientific terms used herein have the same meaning as commonly understood by one of ordinary skill in the art of microbiology, molecular biology, medicinal chemistry, physical chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

In describing and claiming the disclosed subject matter, the following terminology will be used in accordance with the definitions set forth below.

By “administration” or “administering” is meant introducing an ergothioneine (ERGO) based positron emission tomography (PET) probe (or compound), analogs thereof, compositions and pharmaceutical compositions including the ERGO PET probe of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid (e.g., intrathecal), or instillation into body compartments can be used.

As used herein, “alkyl” or “alkyl group” refers to a saturated aliphatic hydrocarbon radical which can be straight or branched and/or substituted or unsubstituted, having 1 to 6 carbon atoms (C1-C6) or 1 to 4 carbon atoms (C1-C4), wherein the stated range of carbon atoms includes each intervening integer individually, as well as sub-ranges. Examples of alkyl include, but are not limited to methyl, ethyl, n-propyl, iso-propyl, n-butyl, sec-butyl, tert-butyl, n-pentyl, and sec-pentyl, and the like.

In accordance with the present disclosure, “a detectably effective amount” of the ERGO PET probe or compositions (e.g., pharmaceutical compositions) including the ERGO PET probe of the present disclosure is defined as an amount sufficient to yield an acceptable image using equipment that is available for clinical use. A detectably effective amount of the ERGO PET probe or compositions including the ERGO PET probe of the present disclosure may be administered in more than one injection. The detectably effective amount of the ERGO PET probe or compositions including the ERGO PET probe of the present disclosure can vary according to factors such as the degree of susceptibility of the individual, the age, sex, and weight of the individual, idiosyncratic responses of the individual, and the like. Detectably effective amounts of the ERGO PET probe or compositions including the ERGO PET probe of the present disclosure can also vary according to instrument and film-related factors. Optimization of such factors is well within the level of skill in the art.

As used herein, the term “subject” includes vertebrates such as humans and mammals (e.g., cats, dogs, horses, etc.). Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a subject. The term “living subject” refers to a subject noted above that is alive and is not dead. The term “living subject” refers to the entire subject and not just a part excised (e.g., a liver or other organ) from the living subject.

The term “detectable” refers to the ability to detect a signal over the background signal.

The term “detectable signal” is a signal derived from non-invasive imaging techniques such as, but not limited to, positron emission tomography (PET). The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

The term “pharmaceutically acceptable carrier” as used herein refers to a diluent, adjuvant, excipient, or vehicle with which the ERGO PET probe or compositions including the ERGO PET probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the ERGO PET probe or compositions including the ERGO PET probe of the disclosure and pharmaceutically acceptable carriers preferably should be sterile. Water is a useful carrier when the ERGO PET probe or compositions including the ERGO PET probe of the disclosure is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the ERGO PET probe, pharmaceutically acceptable salts thereof, with other chemical components, such as physiologically acceptable carriers and excipients. One purpose of a pharmaceutical composition is to facilitate administration of the ERGO PET probe to a subject (e.g., human).

Embodiments of the present disclosure may be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds may be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amounts and a specific type of active ingredient (e.g., the ERGO PET probe or compositions including the ERGO PET probe) in a dosage form may differ depending on various factors. It will be understood, however, that the total daily usage of the ERGO PET probe or compositions including the ERGO PET probe of the present disclosure will be decided by the attending physician or other attending professional within the scope of sound medical judgment. The specific effective dose level for any particular subject will depend upon a variety of factors, including for example, the activity of the specific composition employed; the specific composition employed; the age, body weight, general health, sex, and diet of the subject; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the ERGO PET probe or compositions including the ERGO PET probe at levels lower than those required to achieve the desired effect and to gradually increase the dosage until the desired effect is achieved.

The term “positron emission tomography” as used herein refers to a nuclear medicine imaging technique that produces a three-dimensional image or map of functional/molecular processes in the body. The system detects pairs of gamma rays emitted indirectly by a positron-emitting radioisotope, which is introduced into the body via a molecule specific for a target/process of interest. Images of the target/process of interest in space are then reconstructed by computer analysis. Using statistics collected from tens-of-thousands of coincidence events, a set of simultaneous equations for the total activity of each parcel of tissue can be solved by a number of techniques, and a map of radioactivities as a function of location for parcels or bits of tissue may be constructed and plotted. The resulting map shows the tissues in which the molecular probe has become concentrated. PET technology can be used to trace the biologic pathway of any compound in living humans (and many other species as well), provided it can be radiolabeled with a PET isotope.

The term “label” as used herein refers to any moiety that may be linked (e.g. bonded or otherwise associated with) to the agent (e.g., antibody of TREM-1) of the present disclosure and which may be used to provide a detectable image including, but not limited to, a radiolabel such as a PET probe.

The term “in vivo imaging” as used herein refers to methods or processes in which the structural, functional, molecular, or physiological state of a living being is examinable without the need for a life-ending sacrifice.

The term “non-invasive in vivo imaging” as used herein refers to methods or processes in which the structural, functional, molecular, or physiological state of being is examinable by remote physical probing without the need for breaching the physical integrity of the outer (skin) or inner (accessible orifices) surfaces of the body.

### Discussion:

Embodiments of the present disclosure include compounds and probes such as ergothioneine (ERGO) based positron emission tomography (PET) (ERGO PET) probes or compounds or compositions including the ERGO PET probe such as pharmaceutical compositions, methods of making the ERGO PET probes or compositions, methods of imaging using the ERGO PET probes or compositions, and the like. The present disclosure provides for ERGO PET probes or compositions that can be used for imaging, diagnosing, localizing, monitoring, and/or assessing neurodegenerative diseases and inflammatory diseases or related biological conditions, to monitor the progression (or regression) of the inflammatory diseases, or to assess the response of the inflammatory diseases to treatment, and the like. In an aspect, the neurodegenerative diseases and inflammatory diseases can include, but not limited to, Alzheimer's disease (AD), sepsis, sepsis induced encephalopathy (SIE), multiple sclerosis, epilepsy, traumatic brain injury, cancer, arthritis, inflammatory bowel diseases (e.g., colitis), Huntington's disease, Amytrophic Lateral Sclerosis (ALS), Parkinson's disease, infectious diseases, pain, stroke, chronic fatigue syndrome, depression, schizophrenia, other central nervous system and peripheral inflammatory diseases, or related biological events.

Embodiments of the present disclosure include methods for imaging a sample (e.g., tissue or cell(s)) or a subject, that includes contacting a sample with or administering to a subject ERGO PET probes or compositions and imaging the sample with a PET imaging system. Imaging can be performed in vivo, ex vivo, and/or in vitro. In particular, embodiments of the present disclosure can be used to image neurodegenerative diseases and inflammatory diseases or related biological events. In this regard, the sample or subject can be tested to determine if the sample or subject includes inflammation or related biological conditions, to monitor the progression (or regression) of the inflammation, or to assess the response of the inflammation to treatment, and the like. In an embodiment, the tissue or cells can be within a subject or can have been removed from a subject. In particular, the present disclosure includes methods relating to non-invasive imaging (e.g., using positron emission tomography (PET) imaging system) using the ERGO PET probes or compositions in vivo.

In an embodiment, the ERGO PET probes or compositions can be imaged using imaging systems such as a positron emission tomography (PET) imaging systems (and combined PET/CT and PET/MR systems) or an ex vivo/in vitro phosphor imager. In an embodiment, PET imaging is a preferred embodiment.

In contrast to many other compounds that cannot enter the brain, ERGO PET probes or compositions of the present disclosure are actively transported into the brain by OCTN1 receptors. Since the ERGO PET probes or compositions are actively transported by the OCTN1 receptors, in some embodiments, the method includes mapping OCTN1 receptors within the subject. Additionally or alternatively, in some embodiments, the method includes monitoring concentration in specific regions of the brain and/or body through administration to the subject and imaging of the ERGO PET probes or compositions. For example, in one embodiment, the method includes monitoring concentration of the ERGO PET probes or compositions in the cortex and cerebellum of the subject. In another embodiment, the method includes monitoring concentration of the ERGO PET probes or compositions in the eyes of the subject. In a further embodiment, the method includes monitoring concentration of the ERGO PET probes or compositions in the small intestine.

In addition to being actively transported through the OCTN1 receptors, the ERGO PET compounds disclosed herein bind to and/or react with oxidants and/or amyloid plaques. Accordingly, in some embodiments, the method includes detecting inflammation and/or amyloid plaques using methods as provided herein. As amyloid plaques are implicated in various neurodegenerative diseases, such as Alzheimer's disease, in some embodiments, the method includes detecting and/or diagnosing a subject with a neurodegenerative disease. In such embodiments, the method includes administering the ERGO PET probes or compositions to a subject, monitoring the distribution of the ERGO PET probes or compositions following administration, and diagnosing the subject with a neurodegenerative disease based upon the identification of amyloid plaques. Similarly, the method may include monitoring response to therapy by detecting changes in the presence of amyloid plaques (e.g., increase or decrease in the number or size of plaques).

Furthermore, as ERGO PET probes or compositions disclosed herein may remove oxidants and/or amyloid plaques, in some embodiments, the method includes treating a neurodegenerative disease by administering ERGO PET probes or compositions to a subject in need thereof of a pharmaceutically acceptable dose. In some embodiments, removing the oxidants decreases or prevents the formation of amyloid plaques, thus preventing or slowing the progression of a neurodegenerative disease. Although described herein primarily with respect to neurodegenerative diseases, the disclosure is not so limited and includes other conditions involving oxidants, such as, but not limited to, cancer, cardiovascular disease, and other suitable diseases where antioxidants are beneficial.

Additional details regarding methods of use are provided in Examples 1-3.

The present disclosure provides for ERGO PET probes as well as analogs thereof. The ERGO PET probes or compositions are labeled with a suitable radioisotope, such as, but not limited to, ¹¹C, but optionally substituting H with ¹⁸F, ¹²⁵I, ¹²⁴I, ¹³¹I, ¹²³I, ³²Cl, ³³Cl, ³⁴Cl, ⁷⁴Br, ⁷⁵Br, ⁷⁶Br, ⁷⁷Br, ⁷⁸Br) (e.g., where ¹¹C is not used). In an aspect, the ERGO PET probe is presented by the following structure:

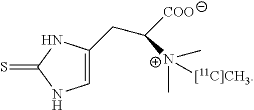

The present disclosure also provides for precursor(s) to the ERGO PET probe(s). In some embodiments, the precursor includes a thiol-histidine molecule with a dimethylamine site available for labeling. For example, in some embodiments, the precursor includes the following structure:

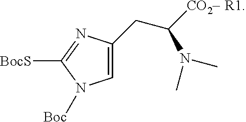

In an aspect, R1 can be a C1-C4 alkyl group such as a methyl group or butyl group. In some embodiments, the functional groups of the thiol-histidine molecule, other than the dimethylamine site, are uniformly blocked with acid-labile protecting groups (e.g., tert-Butyloxycarbonyl protecting group (Bop)).

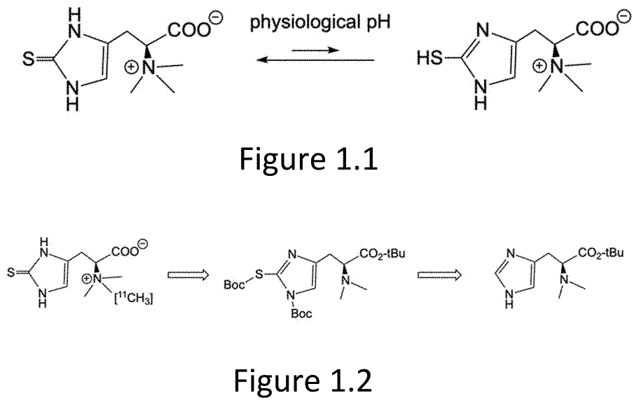

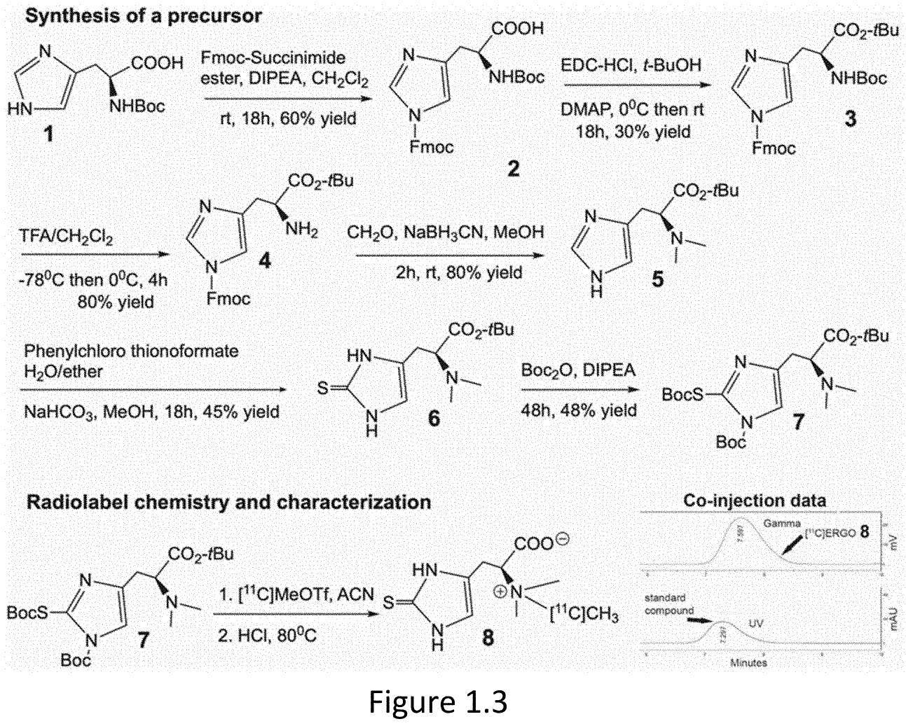

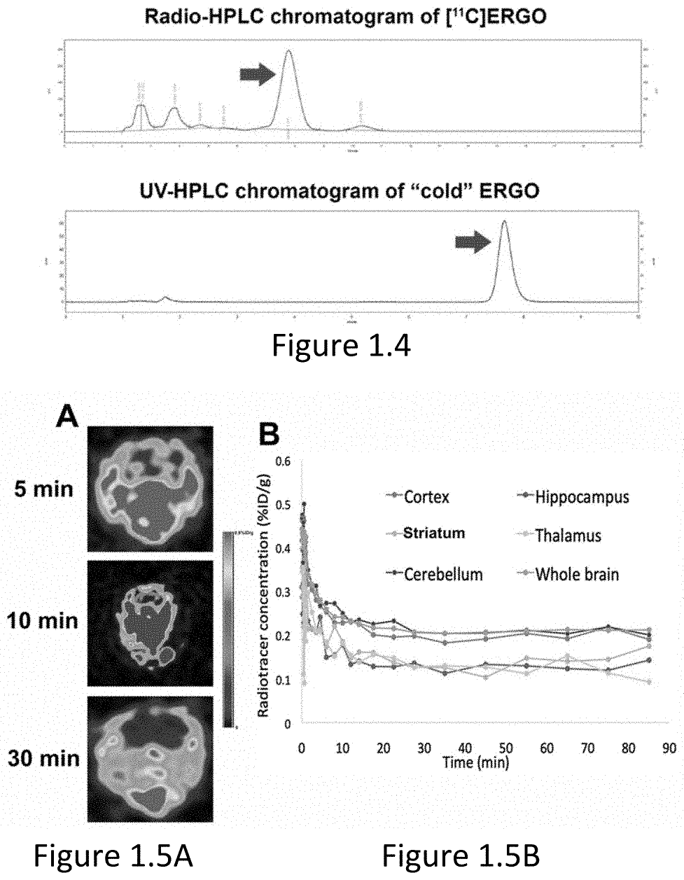

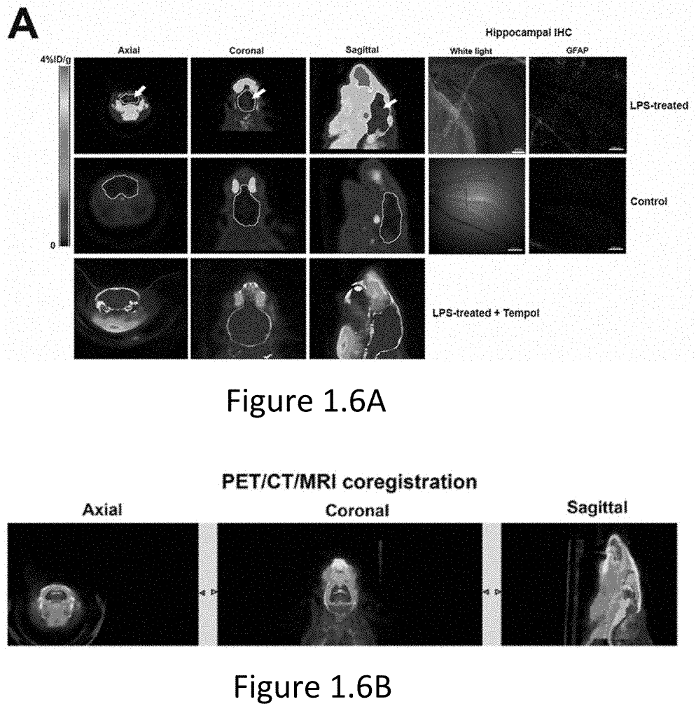

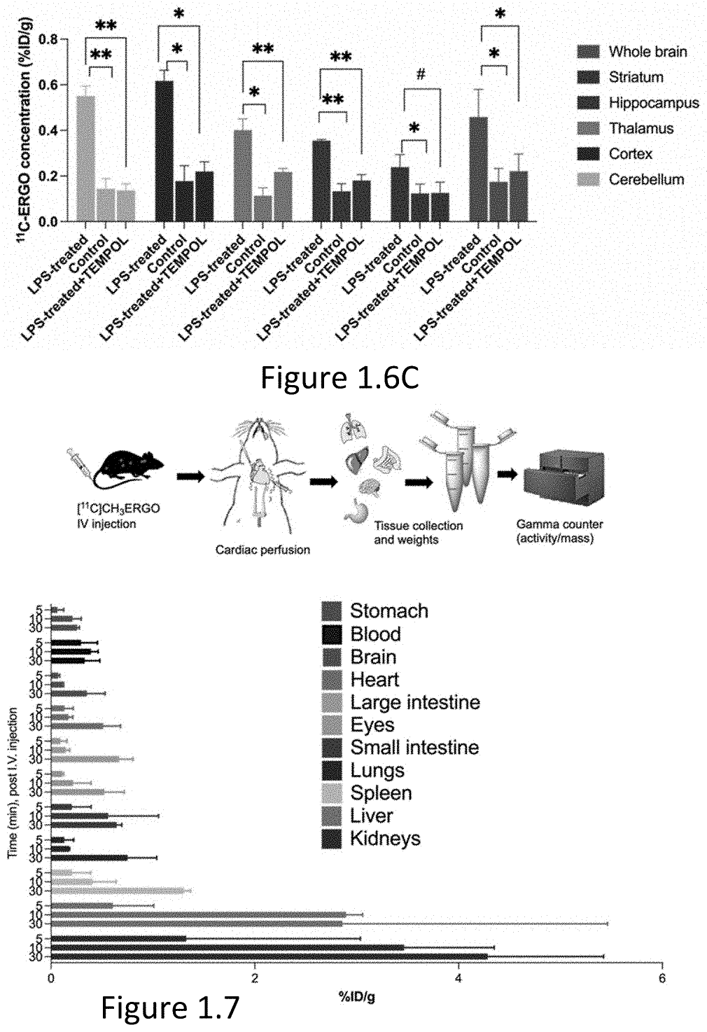

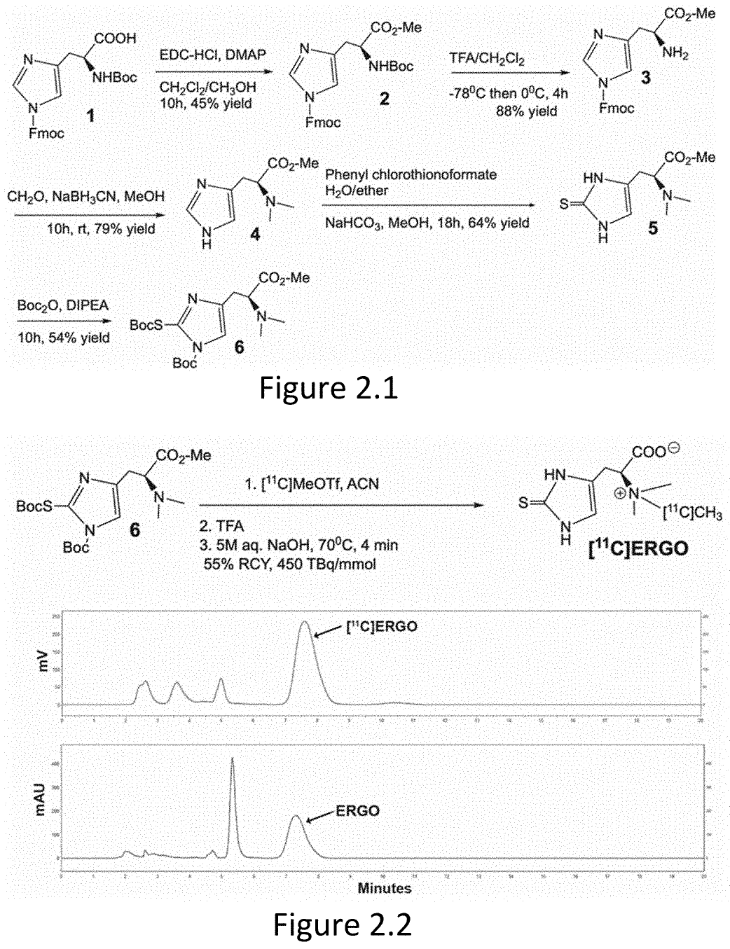

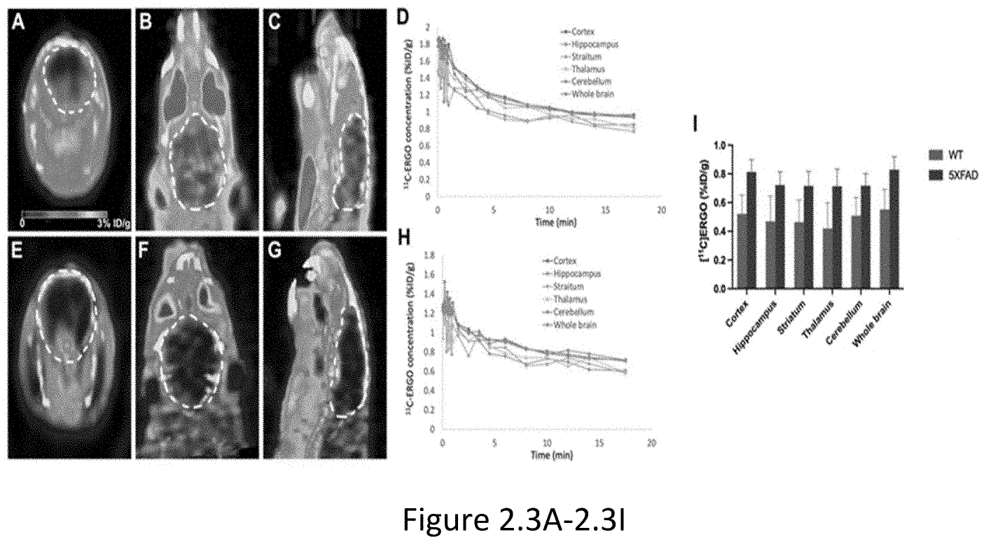

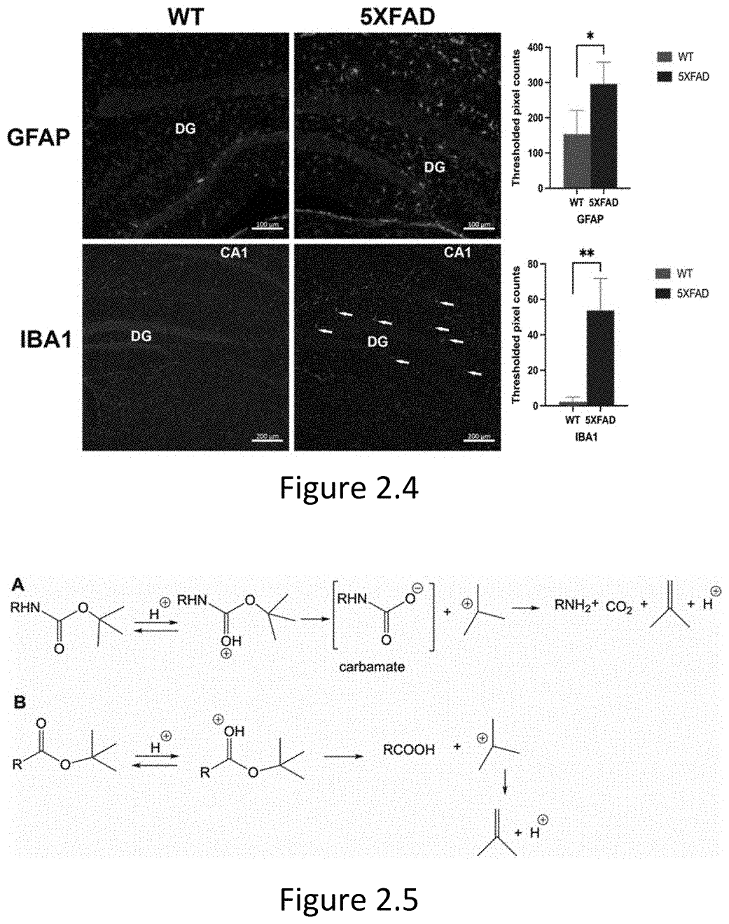

The present disclosure provides for methods of forming the ERGO PET probe. In some embodiments, as illustrated in FIG. 1.3 and FIGS. 2.1 and 2.2. The method shown in FIG. 1.3 is described below (and in Example 1) while the method illustrated in FIGS. 2.1 and 2.2 are described in Example 2, where a difference in each method is that R1 is t-butyl in FIG. 1.3 and methyl in FIGS. 2.1 and 2.2. The method described in FIG. 1.3 includes a three step process of forming intermediate compound 4 from protected histidine 1. Next, intermediate compound 5 is formed from intermediate compound 4 via selective dimethylation of the α-amino group after orthogonal protection of the other functional groups. A solution of intermediate compound 5 in a mixture of water and diethyl ether is then treated with 0-phenylchlorothionoformate in the presence of sodium bicarbonate and stirred overnight at room temperature to furnish thioketone intermediate 6. To form precursor 7, the thioketone intermediate 6 is treated with Boc anhydride, which caps the active functional groups with acid labile groups. Finally, the ERGO PET probe can formed by reacting the precursor 7 with the radiolabel. Additional details are provided in Examples 1 and 2.

In another aspect, the ERGO PET probe is presented by the following structure:

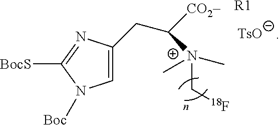

In an aspect, R1 can be a C1-C4 alkyl group such as a methyl group or butyl group and n is 1, 2, or 3. TsO⁻ is a tosylate group. In other aspect, the ¹⁸F can be substituted with ¹²⁵I, ¹²⁴I, ¹³¹I, ¹²³I, ³²Cl, ³³Cl, ³⁴Cl, ⁷⁴Br, ⁷⁵Br, ⁷⁶Br, ⁷⁷Br, or ⁷⁸Br.

In another aspect, the present disclosure also provides for precursor(s) to the ERGO PET probe(s). In some embodiments, the precursor includes the following structure:

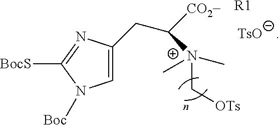

In an aspect, R1 can be a C1-C4 alkyl group such as a methyl group or butyl group and n is 1, 2, or 3.

One method for making the ERGO PET probe including ¹⁸F can include the following:

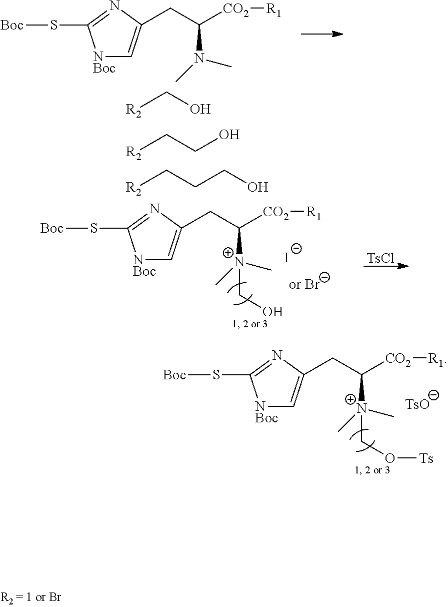

In an aspect, R1 can be a C1-C4 alkyl group such as a methyl group or butyl group and n is 1, 2, or 3. R2 can include halogens such as I or Br. Using dimethylamine derivative as a starting material, where the quaternary amine can be generated by an alkylation reaction using either bromo- or iodoalcohol. The resulting alcohol products enable the creation of a tosylate group, which is a great leaving group for [¹⁸F] labeling. Since the tosylate salt is a very stable solid, this precursor can be handled and stored with convenience (e.g., in a kit of the present disclosure).

Another method for making the precursor(s) to the ERGO PET probe(s) including ¹⁸F is as follows:

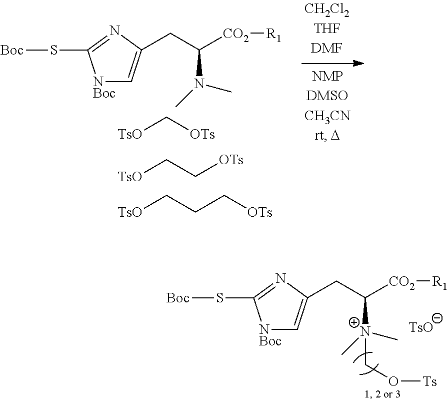

In an aspect, R1 can be a C1-C4 alkyl group such as a methyl group or butyl group and n is 1, 2, or 3. Another chemical method can be employed for making the tosylate precursor. This work focuses on the alkylation of dimethylamine with di-tosylate derivatives. Since the reaction is very robust, slight heating in anhydrous solvents would suffice (e.g., this precursor can be in a kit of the present disclosure).

For either method of making the precursor above, the following reaction can be used to add the ¹⁸F group:

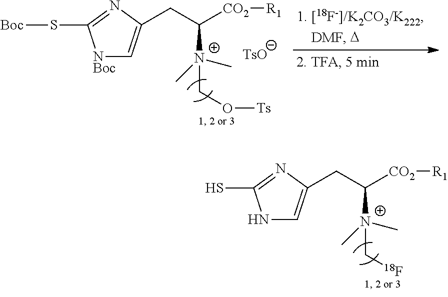

In an aspect, R1 can be a C1-C4 alkyl group such as a methyl group or butyl group and n is 1, 2, or 3. For [¹⁸F] labeling, the tosylate precursor can be dissolved in an anhydrous and polar aprotic solvent, such as DMF or acetonitrile, before dispensing [¹⁸F] fluoride along with potassium carbonate and Kryptofix®222. The temperature of the stirring reaction mixture can be elevated to about 80 to 120° C. for about 20-30 minutes. After labeling, the Boc groups can be removed with trifluoroacetic acid at room temperature in a 5-minute operation to provide the [¹⁸F]ERGO PET radioligand.

In another aspect, the present disclosure also provides compounds having the following structure:

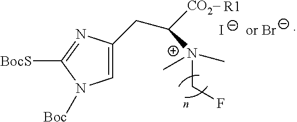

In an aspect, R1 can be a C1-C4 alkyl group such as a methyl group or butyl group and n is 1, 2, or 3. In other embodiments, F is substituted with Br, I, or Cl.

The following provides for a method of making the compound above:

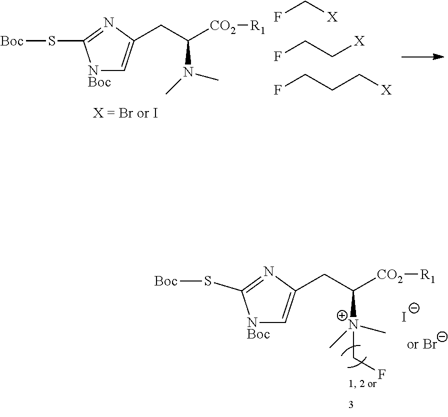

In an aspect, R1 can be a C1-C4 alkyl group such as a methyl group or butyl group and n is 1, 2, or 3. It may be important to develop “cold” compounds, which will assist with the characterization and purification of the [¹⁸F]ERGO PET radioligand. The chemistry for making these compounds can be achieved with the alkylation of dimethylamine starting material with either iodo- or bromofluoromethane or 1-bromo-2-fluoroethane or 1-bromo-3-fluoropropane to afford the corresponding [¹⁹F]“cold” compounds.

### Methods of Use

In general, the ERGO PET probes or compositions can be used in imaging, diagnosing, localizing, monitoring, and/or assessing neurodegenerative disease or inflammatory disease and related biological events as well as tracking response to therapy. Embodiments of the present disclosure can be used to image, detect, study, monitor, evaluate, assess, and/or screen, neurodegenerative disease or inflammatory disease or related biological conditions in vivo or in vitro using the ERGO PET probes or compositions. For example, the ERGO PET probes or compositions provide a detectable signal is emitted that is significantly different from the background signal (e.g., non-inflamed areas) above a certain threshold, indicating the presence of neurodegenerative disease or inflammatory disease, where the threshold depends upon various variables such as the individual subject, the neurodegenerative disease or inflammatory disease, and the like.

In general, the ERGO PET probes or compositions can be used in imaging neurodegenerative disease or inflammatory disease. For example, the ERGO PET probes or compositions is provided or administered to a subject in an amount effective to result in association of ERGO PET probes or compositions with the cells, tissue, or the like associated with the neurodegenerative disease or inflammatory disease. The subject is then introduced to an appropriate imaging system (e.g., PET system) for a certain amount of time (e.g., this depends on radioisotope being used). The ERGO PET probes or compositions becomes associated with cells, tissue, or the like associated with the neurodegenerative disease or inflammatory disease and is detected using the imaging system. The location of the detected signal from the ERGO PET probes or compositions can be correlated with the location of the inflammation associated with the neurodegenerative disease or inflammatory disease. In an embodiment, the dimensions of the location can be determined as well.

In an embodiment, the steps of this method can be repeated at determined intervals so that the location and/or size/stage of the neurodegenerative disease or inflammatory disease can be monitored as a function of time and/or treatment. In particular, the ERGO PET probes or compositions can find use in a subject undergoing treatment (e.g., using a drug, etc.), to aid in visualizing the response of the neurodegenerative disease or inflammatory disease to the treatment. In this embodiment, the ERGO PET probes or compositions is typically visualized and the imaging signal is quantified prior to treatment, and periodically (e.g., daily, weekly, monthly, intervals in between these, and the like) during treatment, and the like, to monitor the regions and extent of the neurodegenerative disease or inflammatory disease.

An embodiment of the present disclosure can include a method for diagnosing the presence of neurodegenerative disease or inflammatory disease that includes: administering to a subject a ERGO PET probes or compositions in a detectably effective amount, wherein the ERGO PET probes or compositions are described herein; imaging at least a portion of the subject; and detecting the ERGO PET probes or compositions, wherein the location of the ERGO PET probes or compositions corresponds to neurodegenerative disease or inflammatory disease corresponding to the neurodegenerative disease or inflammatory disease, where detection of the ERGO PET probes or compositions in a location above a threshold is an indication of presence of the neurodegenerative disease or inflammatory disease at the location.

In an embodiment, the method can be used to select appropriate patients to receive therapy. For example, a patient can be given a selected drug, and the neurodegenerative disease or inflammatory disease can be measured to determine if the drug is having the desired results in the patient.

It should be noted that the amount effective to result in uptake or association of the ERGO PET probes or compositions into the cells or tissue of interest may depend upon a variety of factors, including for example, the age, body weight, general health, sex, and diet of the subject; the time of administration; the route of administration; the rate of excretion of the specific probe employed; the duration of the treatment; the existence of other drugs used in combination or coincidental with the specific composition employed; and like factors, well known in the medical arts.

### Kits

The present disclosure also provides packaged compositions or pharmaceutical compositions comprising a pharmaceutically acceptable carrier and precursor compounds and/or probes (e.g., precursor compounds for the ERGO PET probes of the disclosure). In certain embodiments, the packaged compositions or pharmaceutical composition includes the reaction precursors and precursor compounds, as provided herein, to be used to generate the ERGO PET probes or compositions according to the present disclosure. Other packaged compositions or pharmaceutical compositions provided by the present disclosure further include materials including at least one of: instructions for making the ERGO PET probes from the precursor compounds, instructions for using the ERGO PET probes or compositions to image a subject, or subject samples (e.g., cells or tissues), which can be used as an indicator of relevant conditions, diseases, and/or biological related conditions as well as other uses described herein.

### Dosage Forms

Embodiments of the present disclosure can be included in one or more of the dosage forms mentioned herein. Unit dosage forms of the pharmaceutical compositions (the “composition” includes at least ERGO PET probes or compositions of the present disclosure) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the ERGO PET probes or compositions, pharmaceutically acceptable salts thereof, with other chemical components, such as physiologically acceptable carriers and excipients. One purpose of a pharmaceutical composition is to facilitate administration of the ERGO PET probes or compositions to a subject (e.g., human).

Embodiments of the present disclosure may be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an active compound may be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amounts and a specific type of active ingredient (e.g., ERGO PET probes or compositions) in a dosage form may differ depending on various factors. It will be understood, however, that the total daily usage of the compositions of the present disclosure will be decided by the attending physician or other attending professional within the scope of sound medical judgment. The specific effective dose level for any particular subject will depend upon a variety of factors, including for example, the activity of the specific composition employed; the specific composition employed; the age, body weight, general health, sex, and diet of the subject; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

This Example discusses the development and validation of Ergothioneine (ERGO) PET radioligands. ERGO is a rare amino acid mostly found in fungi, including mushrooms, with recognized antioxidant activity to protect tissues from damage by reactive oxygen species (ROS) components. The biodistribution of ERGO has previously been performed solely in vitro using extracted tissues. This Example discusses the development of a feasible chemistry for the synthesis of an ERGO PET radioligand, [¹¹C]ERGO, to facilitate in vivo study. The radioligand probe was synthesized with identical structure to ERGO by employing an orthogonal protection/deprotection approach. [¹¹C]methylation of the precursor was performed via [¹¹C]CH₃OTf to provide [¹¹C]ERGO radioligand. The [¹¹C]ERGO was isolated by RP-HPLC with a molar activity of 690 TBq/mmol.

To demonstrate the biodistribution of the radioligand, approximately 37 MBq/0.1 mL was administered in 5XFAD mice, a mouse model of Alzheimer's disease, via the tail vein. The distribution of ERGO in the brain was monitored using 90-minute dynamic PET scans. The delivery and specific retention of [¹¹C]ERGO in an LPS-mediated neuroinflammation mouse model was also demonstrated. For the pharmacokinetic study, the concentration of the compound in the serum started to decrease 10 min after injection while starting to distribute in other peripheral tissues. In particular, a significant amount of the compound was found in the eyes and small intestine. The radioligand was also distributed in several regions of the brain of 5XFAD mice, and the signal remained strong 30 min post-injection. This is the first time the biodistribution of this antioxidant and rare amino acid has been demonstrated in a preclinical mouse model in a highly sensitive and non-invasive manner.

### Introduction

L-ergothioneine (ERGO) is a food-derived hydrophilic antioxidant available in fungi and various bacteria, but not in animals or higher plants. It has been known as an antioxidant since its discovery a century ago from fungi of the genus Claviceps purpurea. Chemically, this rare betaine-based amino acid has a similar chemical structure to histidine but with the presence of a sulfhydryl moiety on the imidazole ring. The molecule exists as a tautomeric form between thioketone and thiol derivatives (FIG. 1.1), albeit the former predominates at physiological pH. Because plants and animals cannot produce ERGO, it must be obtained from the diet. Mushrooms, in particular are a rich source of ERGO. As an antioxidant agent ERGO is capable of preventing cell and tissue damage, a key contributor to aging, by protecting against free radicals and oxidative stress. Its acquired adaptive antioxidant capability for the protection of injured tissues could be the reason for the observations that the highest concentration of ERGO are usually found in the red blood cells of old-age individuals, brain, liver, kidney, ocular tissues, and injured tissues. Additional evidence suggests that ERGO could target mitochondria and dampen the excess of mitochondria-specific ROS in response to oxidative stress.

ERGO is also implicated in a number of neurological pathways. Substantial research data indicate that ERGO is a physiological antioxidant cytoprotectant. Protection against cytotoxicity elicited by Cu (II), hydrogen peroxide, iron, and sodium nitrite is derived from the conspicuous affinity of ERGO for metal cations, such as Fe and Cu, permitting capture and neutralization of associated radicals. It has been demonstrated that ERGO concentration decreases significantly with age, and markedly lower levels were found in individuals with mild cognitive impairment compared to the controls, supporting the potential for ERGO deficiency to act as a risk factor for neurodegeneration. It is worthwhile to mention that ERGO does not permeate the blood-brain barrier (BBB); its uptake in cells is mediated by an OCTN1 (organic cation transporter novel, type 1) receptor. Other studies have shown that ERGO can protect neurons both in vitro and in vivo against a spectrum of stressors. Taken altogether, these data strongly suggest that ERGO is involved in healthy aging, serving as a “longevity vitamin”. The term “vitamin” is used in here since ERGO is a micronutrient, that the body does not need a lot of to function, but the material needs to be constantly available by uptake from an external source such as foods or supplements and it therefore does fit into that category.

To date, all preclinical studies had relied solely on in vitro analysis of ERGO using analytical and bioassay methods for analyzing the ERGO distribution from extracted tissues. The same holds true for clinical studies to validate the benefit of ERGO consumption in the diet; serum samples were collected for assessing the ERGO levels. This necessitated the development of an ERGO probe to facilitate non-invasive, real-time, and repeated imaging of ERGO distribution and pharmacokinetics in preclinical and clinical settings. To this end, report herein for the first time is the development of a [¹¹C]ERGO radioligand. Further, this Example demonstrates that this antioxidant could be distributed and retained in the brain of a mouse model of Alzheimer's disease. This observation was further confirmed using a mouse model of LPS-induced neuroinflammation. The proof-of-principle PET imaging in this Example showed that ERGO is distributed across several subregions in the brains, albeit with much higher retention in pathological brains than normal counterparts. Interestingly, the whole-body biodistribution study showed that ERGO is also distributed significantly in the eyes and small intestine.

### Materials and Methods

TEMPOL was acquired from Tocris Bioscience (Fisher Scientific). Animal experiments were conducted per the guidelines established by the Vanderbilt University's Institutional Animal Care and Use Committee (IACUC) and the Division of Animal Care. The performed work was approved by Vanderbilt IACUC with an extended protocol, M1700044-01. In addition, all of the works involving live animals were compliant with the ARRIVE guidelines. In a typical imaging procedure, anesthetized mice received 2% of isoflurane via inhalation, supplied with oxygen using a precision vaporizer.

### General Synthesis

All commercially available reagents and solvents were used as received. L-(+)-Ergothionene was purchased from Cayman Chemicals. Reactions were monitored by a Agilent LC/MS 1260 Infinity II. Products were purified using a Telodyne Combiflash Rf automated purification instrument using normal phase or reverse phase. NMR spectra were recorded on a 400 MHz Bruker AVANCE 400 equipped with a 9.3 T Oxford Magnet or 600 MHz Bruker AVIII console equipped with a 14.1 T Bruker Magnet. 1H NMR chemical shifts were referenced to the residual solvent signal; 13C NMR chemical shifts were referenced to the deuterated solvent signal. Data are presented as follows: chemical shift d (ppm), multiplicity (s=singlet, d=doublet, dd=doublet of doublet, t=triplet, m=multiplet).

### Synthesis of the PET precursor (FIG. 1.3)

1. N^{T}-M9H-fluoren-9-yl)methoxy)carbonyl)-N^{α}-(tert-butoxycarbonyl)-L-histidine 2. To a stirring solution of histidine amino acid 1 (1.0 g, 3.9 mmol), FMOC-succinimide (1.45 g, 4.3 mmol), and DIPEA (0.605 g, 4.7 mmol) in methylene chloride (50 mL) and stirred overnight. Reaction was diluted with water and methylene chloride. The product was extracted 3× with methylene chloride. The organic layers were combined, washed with 1M HCl, saturated sodium bicarbonate, brine, dried over sodium sulfate, filtered and concentrated under reduced pressure to provide a desired product with 60% yield.

¹H CDCl₃ (400.13 mHz): 8.26 (d, J=1.2 Hz, 1H); 7.79 (d, J=7.6 Hz, 2H); 7.56 (d, J=7.6 Hz, 2H); 7.43, (t, J=7.2 Hz, 2H); 7.34 (t, J=7.2 Hz, 2H); 7.24 (s, 1H); 5.51 (d, J=6.0 Hz, 1H); 4.70 (dd, J₁=7.2 Hz, J₂=1.6 Hz, 2H); 4.56 (t, J=7.6 Hz, 1H); 4.35 (t, J=6.8 Hz, 1H); 3.32 (dd, J₁=14.8 Hz, J₂=2.8 Hz, 1H); 3.20 (dd, J₁=14.4 Hz, J₂=5.2 Hz, 1H); 1.47 (s, 9H). ¹³C CDCl₃ (100.6 mHz): 172.9, 168.6, 155.4, 148.1, 142.7, 141.5, 137.5, 128.4, 127.6, 124.9, 120.5, 115.6, 80.0, 70.6, 53.6, 52.8, 46.6, 29.9, 28.5.

2. (9H-fluoren-9-yl)methyl(S)-4-(3-(tert-butoxy)-2-((tert-butoxycarbonyl)amino)-3-oxopropyl)-1H-imidazole-1-carboxylate 3. To a stirring solution of 2 (3.90 mmol) in methylene chloride (50 mL) and tert-butanol (10 mL) at 0 C was added: EDC-HCl (744 mg, 3.9 mmol), and DMAP (95 mg, 0.78 mmol. This solution was allowed to warm up to r.t. and stirred overnight. Reaction was diluted with water and methylene chloride. The product was extracted 3× with methylene chloride. The organic layers were combined, washed with 1M HCl, saturated sodium bicarbonate, brine, dried over sodium sulfate, filtered and concentrated under reduced pressure to provide the desired product (12% yield).

¹H CDCl₃ (400.13 mHz): 7.99 (s, 1H); 7.81 (d, J=7.6 Hz, 2H); 7.58 (d, J=7.6 Hz, 2H); 7.48, (t, J=7.2 Hz, 2H); 7.35 (t, J=7.2 Hz, 2H); 7.17 (s, 1H); 5.03 (m, 1H); 4.72 (dd, J₁=6.4 Hz, J₂=2.0 Hz, 2H); 4.20 (m, 1H); 3.03 (m, 2H); 1.44 (s, 9H).

3. (9H-fluoren-9-yl)methyl (S)-4-(2-amino-3-(tert-butoxy)-3-oxopropyl)-1H-imidazole-1-carboxylate 4. To a stirring solution of 3 (3.90 mmol) in methylene chloride at −78° C. was added TFA (5 mL) dropwise. This solution was allowed to warm up to 0° C. and stirred until completion. The solution was then neutralized with sodium bicarbonate and diluted with water and methylene chloride. The product was extracted 3× with methylene chloride. The organic layers were combined, washed with brine, dried over sodium sulfate, filtered and concentrated under reduced pressure to provide the desired product with 63% yield.

¹H CDCl₃ (400.13 mHz): 7.99 (s, 1H); 7.77 (d, J=7.6 Hz, 2H); 7.57 (d, J=6.8 Hz, 2H); 7.41, (t, J=6.0 Hz, 2H); 7.33 (t, J=7.6 Hz, 2H); 7.14 (s, 1H); 5.03 (m, 1H); 4.59 (m, 2H); 4.18 (m, 1H); 3.05 (m, 2H); 1.38 (s, 9H).

4. tert-butyl N^{α}N^{α}-dimethyl-L-histidinate 5. To a stirring solution of 4 in MeOH (20 mL) was added NaBH₃CN (362 mg, 5.8 mmol) and CH₂O (37% in H₂O, 702 mg, 23.4 mmol). The reaction was capped and stirred for 2 hrs. The resulting solution was then concentrated via rotovap. The crude oil was purified by reverse phase chromatography. The product was verified by LC/MS and used as is in the next step with a 82% yield. The Fmoc group was removed in this step through interaction with the NaBH₃CN to provide the desired product.

¹H D20 (400.13 mHz): 7.47 (s, 1H); 6.83 (s, 1H); 4.79 (m, 1H); 4.29 (m, 2H); 2.58 (s, 6H); 1.24 (s, 9H).

5. tert-butyl (S)-2-(dimethylamino)-3-(2-thioxo-2,3-dihydro-1H-imidazol-4-yl)propanoate 6. To a stirring solution of 5 (31.5 mg, 0.13 mmol) in water (2 mL) and diethyl ether (2 mL) was added sodium bicarbonate (65 mg, 0.78 mmol), and phenylchloro thionoformate (24.7 mg, 0.14 mmol) and stirred overnight. Reaction was diluted with water and diethyl ether. The product was extracted 3× with diethyl ether. The organic layers were combined, washed with brine, dried over sodium sulfate, filtered and concentrated under reduced pressure. The resulting oil was then redissolved in MeOH (5 mL) and triethylamine (55 uL) was added. This solution was stirred overnight. The resulting solution was then concentrated under reduced pressure. Product was purified by reverse phase chromatography to provide a final product (16.0 mg) 45% yield. And the product was immediately used for the next step.

6. tert-butyl (S)-4-(3-(tert-butoxy)-2-(dimethylamino)-3-oxopropyl)-2-((tert-butoxycarbonyl)thio)-1H-imidazole-1-carboxylate 7. To a stirring solution of 6 (16.0 mg, 0.066 mmol) in methylene chloride (10 mL) was added: Boc anhydride (32 mg, 0.145 mmol), and DIPEA (19 mg, 0.145 mmol) and stirred over the course of 48 h. The resulting solution was concentrated and purified by flash chromatography (0-50% CH₂Cl₂/(20% MeOH/CH₂Cl₂)) to afford the precursor 7 (15.0 mg) with 48% yield.

¹H CDCl₃ (600.13 mHz): 7.16 (s, 1H); 3.40 (m, 1H); 2.86 (dd, J₁=14.4 Hz, J₂=8.4 Hz, 1H); 2.72 (dd, J₁=14.4 Hz, J₂=6.6 Hz, 1H); 2.28 (s, 6H); 1.48 (s, 9H); 1.37 (s, 9H); 1.32 (s, 9H). ¹³C CDCl₃ (150.9 mHz): 170.9, 165.3, 146.7, 139.9, 135.1, 119.8, 86.8, 85.8, 81.2, 67.3, 41.8, 29.8, 28.6, 28.3, 28.2, 27.9, 22.3, 22.0.

MS: calculated: 471.2403, detected: 471.1845.

### [¹¹C]ERGO Radiotracer Synthesis

The [¹¹C]CO₂ was made by irradiating a target filled with nitrogen and 1% oxygen gas with protons. The [¹¹C]CO₂ was then trapped on nickel Shimalite with molecular sieves at room temperature. The [¹¹C]CO₂ was then converted to [¹¹C]CH₄ by heating the trapped [¹¹C]CO₂ to 400° C. in the presence of hydrogen gas. The [¹¹C]CH₄ was then released from the nickel Shimalite at 400° C. and isolated on molecular sieves at −75° C. The [¹¹C]CH₄ was then converted to [¹¹C]MeI via a recirculation through gaseous iodine at ˜720° C., with the [¹¹C]MeI being trapped on Porapak N with each cycle. The [¹¹C]MeI was then released from the Porapak N by heating with a gentle flow of helium that is passed through an AgOTf impregnated column at ˜200° C. to convert the [¹¹C]MeI to [¹¹C]MeOTf. This [¹¹C]MeOTf was bubbled into a solution of precursor in 250 μl acetonitrile at −10° C. After the activity transfer was complete, the reaction mixture was heated to 80° C. for 2 minutes. At this time, hydrochloric acid (6M, 250 μl) was added, and the reaction mixture was heated at 70° C. for 5 minutes, cooled to room temperature, and diluted with water (1 mL). The reaction mixture was passed through an ion-retardation resin (Ag11-A8, 3 g) into the product vial, and the resin was rinsed with water (5 mL) into the product vial. The product was then transferred to the final vial, and an aliquot was removed for quality control analysis.

The radiochemical purity and the identity of the [¹¹C]ERGO were characterized using an analytical HPLC system, equipped with a UV absorption detector (λ=254 nm) and a radio-detector (Bioscan Flow-Count). The chromatography setup included a SeQuant ZIC-HILIC 150×4.6 mm with a typical mobile phase of acetonitrile (75%) in water at a flow rate of 1 mL/min. The identity of the [¹¹C]ERGO was confirmed by comparing the retention time with co-injected and unlabeled ERGO along with the gamma peak. The molar activity of the radioligand is 690 TBq/mmol.

### PET/CT Data Processing and Analysis

The dynamic acquisition was divided into twelve 5 sec frames, four 60 sec frames, five 120 sec frames, three 5 min frames, and six 10 min scans. The data from all possible lines of response (LOR) were saved in the list mode raw data format. The raw data were then binned into 3D sinograms with a span of 3 and ring difference of 47. The images were reconstructed into transaxial slices (128×128×159) with voxel sizes of 0.0815×0.0815×0.0796 cm³, using the MAP algorithm with 16 subsets, 4 iterations, and a beta of 0.0468. For anatomical co-registration, immediately following the PET scans, the mice received a CT scan in a NanoSPECT/CT (Mediso, Wash. D.C.) at an X-ray beam intensity of 90 mAs and x-ray peak voltage of 45 kVp. The CT images were reconstructed into 170×170×186 voxels at a voxel size of 0.4×0.4×0.4 mm³. The PET/CT images were uploaded into Amide software (www.sourceforge.com), co-registered to an MRI template made in-house, and volumetric regions-of-interest were drawn around the cortex, hippocampus, striatum, thalamus, and cerebellum in addition to the whole brain. The PET images were normalized to the injected dose, and the time-activity-curves (TACs) of the mean activity within the ROIs were estimated for the entire duration of the scans.

### Static PET Scan.

The mice were injected with ˜20 MBq/0.1 ml 11C-ERGO via the tail vein and returned to their cages for 10 min. Then they were anesthetized with 2% isofluorane and imaged in an Inveon microPET (Siemens Pre-clinical, Knoxville, Tenn., USA) for 30 min in static mode followed by a CT scan using a NanoSPECT/CT (Mediso, Wash. D.C.) at an x-ray beam intensity of 90 ms and x-ray voltage of 45 kVp. PET images were reconstructed using the iterative MAP reconstruction algorithm with 18 iterations and a beta smoothing value of 0.001 into 128×128×95 slices with a voxel size of 0.388 mm×0.388 mm×0.796 mm. The CT images were reconstructed into 170×170×126 slices at a voxel size of 0.4×0.4×0.4 mm\hat{ }3. The PET and CT images were co-registered using the imaging tool AMIDE.

### Cardiac Perfusion Procedure and Tissue Collection

At 5 or 10 min post injection, anesthetized mice were laid on the shallow tray filled with crushed ice and the thoracic cavity was accessed using a scalpel after making 5-6 cm mid-line incision from the abdominal area. After careful separation of the liver from the diaphragm, the thoracic opening was stabilized with a retractor. Perfusion was performed as we described in the past. Following removal of air bubbles, approximately 30 mL (pH7.4) of ice-cold PBS buffer is slowly injected in the left ventricle toward the ascending aorta using a 25-G syringe while the right atrium was snipped off using a curved point squeeze snip scissors to facilitate drainage of the systemic venous return. Then, 30 mL of 4% paraformaldehyde (PFA, pH 7.4) was perfused. When completed, tissues were harvested and weighed before counting using the automatic gamma counter (Hidex).

### Animal Models

5XFAD and control C57BL/6J mice were maintained at Vanderbilt University under standard conditions, in a 12-h light/dark cycle, and with free access to food and water. The 5XFAD mice over express both mutant human amyloid precursor protein (APP) and presenilin 1 (PS1), correlating with high burden and accelerated accumulation of the A13. A colony of 5XFAD transgenic mice obtained from Jackson Laboratories was maintained by crossing 5XFAD mice with a WT C57BL/6J strain. The 5XFAD mice were maintained as heterozygous.

The mouse model of LPS-induced neuroinflammation was developed based on past reports. The LPS derived from Escherichia coli 0111:64 (Sigma Aldrich, St Louis, Mo.) was formulated in sterilized dd. water and given a high dose of LPS formulation (5 mg/Kg) through intraperitoneal injection 24 h before PET imaging. This high dose would result in approximately 12-13% body weight loss over the course of 24 h. After PET imaging, animals were sacrificed, and the brains were collected for histology analysis.

### Immunohistochemistry

Brains embedded in OCT were cut into sagittal sections (10 μm) using a Tissue-Tek cryostat and mounted onto charged glass slides. Prior to staining, slides were washed with PBS (10 min); then, they were treated with blocking buffer (5% normal goat serum, 0.2% Triton X-100, 0.5% bovine albumin in PBS) for 1 h at room temperature. The treated sections were then incubated overnight at 4° C. with primary anti-GFAP antibody (1:100 dilution, Biolegend San Diego, Calif., USA). Slides were washed with PBS (3×) for 10 min each, the sections were subsequently incubated with secondary antibody goat anti-mouse Alexa Fluor 488 (1:200 dilution, Thermo Fisher Scientific, Carlsbad, Calif., USA) for 30 min at room temperature. The sections were then washed with PBS twice for 10 minutes and once for 30 minutes, and cover slipped with an antifade mounting medium (Vector Laboratories, Burlingame, Calif.) before observation under a fluorescence microscope.

### Statistical Analysis.

Unpaired t-test was used to compare the mean signal (% ID/g) difference between two independent subjects. A P value of ≤0.05 was considered as a statistically significant difference.

### Results

### Retrosynthesis Analysis and Development of Precursor

Retrosynthetic analysis of [¹¹C]CH₃ labeled ERGO suggests the precursor should have the thiol-histidine molecule with the dimethylamine site available for labeling, while the rest of other functional groups should be uniformly blocked with acid-labile protection groups (FIG. 1.2). They can thus be removed simultaneously as quickly as possible given the short half-life of [¹¹C]CH₃. The next major transformation involves the insertion of the thiol group into the imidazole ring.

FIG. 1.3 describes the successful synthesis of [¹¹C]ERGO PET radiotracer 8. The preparation of intermediate 4 would take three steps starting from protected histidine 1 to set the stage for the crucial synthesis of compound 5. The formation of key intermediate 5 via dimethylation of the α-amino group was achieved selectively after orthogonal protection of the other functional groups. When the solution of 5 in a mixture of water and diethyl ether was treated with 0-phenylchloro thionoformate in the presence of sodium bicarbonate and stirred overnight at room temperature to furnish thioketone intermediate 6. Finally, the precursor 7 was synthesized by treating compound 6 with Boc anhydride to ensure all the active functional group are capped with acid labile groups.

With the precursor available, next, we labeled the ERGO precursor 7 with [¹¹C]CH₃ using a commercial automated radiosynthesis module (GE TRACERlab FXc-Pro). The [¹¹C]CO₂ was converted to [¹¹C]MeOTf using the standard reaction conditions, reacted with precursor 7 at 80° C., and subsequently deprotected under acidic conditions to yield [¹¹C]ERGO 8 (Supplementary data, FIG. 1.4). The [¹¹C]ERGO was pH adjusted by passage through an ion-retardation resin and used without further purification. The product was produced with an excellent molar activity of 690 TBq/mmol.

### In Vivo Dynamic PET Scanning of [¹¹C]ERGO Biodistribution in the Brain

To test the distribution of ERGO non-invasively in vivo, approximately 37 MBq/0.1 mL of [¹¹C]ERGO probe was given to anesthetized 5XFAD mice (8-month-old, n=4) in single bolus intravenous (I.V.) injection via the lateral tail vein. The administration of the probe was simultaneous with the start of a 90-minute dynamic PET imaging, followed by subsequent CT scan. The PET images were normalized to the injected dose, and the time-activity-curves (TACs) of the mean activity within the ROIs were estimated for the entire duration of the scans. Within 5 min post I.V. injection, [¹¹C]ERGO can be detected in the brain parenchyma (FIG. 1.5A, top panel). Approximately 10 min post I.V. injection, the radioligand was distributed in several areas of the brain (FIG. 1.5A, middle panel), and it appeared the probe still remained in the brain 30 min post radiotracer injection (FIG. 1.5A, bottom panel). Using an age-matched mouse brain template, the positron-emitted signal at different brain regions was quantified using AMIDE software. The data showed that the probe was distributed with a high concentration in the cerebellum and cortex (FIG. 1.5B). Nevertheless, the presence of the probe in other brain areas, such as the hippocampus, striatum, and thalamus, was also significant.

### Imaging LPS-Induced Neuroinflammation Using [¹¹C]ERGO Radioligand

The reported LPS mouse model of neuroinflammation in the past [40] was reproduced for the validation of the specificity of the probe. Approximately 24 h after injection of WT mouse with LPS (5 mg/Kg) via intraperitoneal injection, the animals showed signs of withdrawing and lacked grooming. The animals experienced noted body weight loss over 10% within a day after treatment. Further, histological analysis of the brain sections at the hippocampal region showed a significant upregulation of GFAP in the LPS-treated mice (FIG. 1.6A). The control nontreated WT (n=3) and LPS-treated WT mice (n=3) were injected with [¹¹C]ERGO radioligand (20 MBq/0.1 mL) via I.V. injection. For the blocking study, another cohort of LPS-treated WT mice (n=3) was injected with Tempol (4-hydroxy-2,2,6,6-tetramethyl piperidinoxyl) (0.2 mg/mL) prior to injection of the same dose of [¹¹C]ERGO. Ten minutes after injection, each animal was imaged for a period of 30 min, followed by CT. Data shown in FIG. 1.6A indicated that ERGO was distributed and retained in the brain of LPS-treated mice resulting in higher signal intensity on the PET scans. The PET information was coregistered with CT and MRI using the MRI template of age-matched mice. When PET signal in each brain region was analyzed and compared (Supplementary data, FIG. 1.6B), the data showed that the signal from LPS-treated mice was stronger than that from nontreated counterparts across every major region of the brain (FIG. 1.6C). The specificity of the probe was demonstrated in the blocking study; as co-injection of the [¹¹C]ERGO probe with the excess amount of Tempol resulted in a significant reduction of the PET signal (FIGS. 1.6A-C).

### Whole-Body Biodistribution of [¹¹C]ERGO Radioligand

Cohorts of WT mice were injected with [¹¹C]ERGO probe, followed by cardiac perfusion 5 (n=5), 10 (n=5) and 30 (n=6) minutes after injection before tissues were collected for activity counting. The data in FIG. 1.7 showed that ERGO was quickly distributed to every organs, including those have barriers such as brain (blood-brain barrier) and eyes (blood-retinal barrier) 5 minutes post I.V. injection. About 30 minutes after injection of [¹¹C]ERGO, the concentration in the blood decreased (from 0.39% ID/g to 0.33% ID/g), at the same time compound continued to accumulate in the peripheral tissues, including the brain (from 0.13% ID/g to 0.35% ID/g), the eyes (from 0.11% ID/g to 0.52% ID/g), spleen (from 0.2% ID/g to 0.97% ID/g), small intestine (from 0.2% ID/g to 0.64% ID/g), heart (from 0.13% ID/g to 0.51% ID/g), kidneys (from 1.33% ID/g to 4.2% ID/g) and liver (0.61% ID/g to 2.86% ID/g).

### Discussion

One of the objectives of this probe development was to assess a number of synthetic pathways to ensure the probe would be designed in a way that minimized the potential alternation of the intrinsic chemical structure. Ideally, we sought to maintain an identical structure to ERGO. In this respect, the most logical approach focuses on the quaternary amine, which presents a convenient opportunity to insert the [¹¹C]CH₃ radioisotope through the use of an [¹¹C]CH₃ electrophile. Since ERGO comprises four distinct functional groups and each can be activated as a strong nucleophile at their respective pKa, so it is conceivable that each step of the chemistry may require prior protection and deprotection using orthogonal strategy. Further, incorporating these protecting groups is crucial to enhance the hydrophobicity of this water-soluble zwitterion molecule for organic solvents. Given that ERGO and histidine share an overall structural similarity; therefore, taking all of these observations into an account, the design suggests the adoption of either N- or C-terminal protected histidine as the starting material.

Multiple attempts were tried to synthesize the precursor 7; in almost every attempt, two major issues were encountered. First, the product was so polarized that it proved to be challenging for purification using flash chromatography. Second, the protecting groups were sometimes so bulky that they obscured the next step in the process, or they were cleaved off. Therefore, a protecting group must be carefully selected for the successful synthesis. As in this case, using an orthogonal strategy, the Fmoc group can be removed without affecting the Boc group in the following steps. Aside from protection, the Fmoc helps to enhance hydrophobicity and its UV active nature, enables reaction monitoring. After three functional groups were protected, the Boc from the N-terminal region was selectively removed using TFA in methylene chloride at −78° C. to afford 4 (FIG. 1.3). The reaction was carefully performed and monitored to ensure the t-Bu ester is tolerant to these reaction conditions. There was no sign that t-Bu ester was affected for an extension of up to 4 hours. However, when the reaction was continued overnight, both the Boc and the t-Bu ester groups were removed completely. It is interesting to mention that when treating compound 4 with sodium cyanoborohydride during the reductive amination, the Fmoc group was also removed concomitantly. This was an unintentional but necessary process. Fabrication of an appropriate PET precursor requires that all protecting groups should have a Boc group, which can be removed simultaneously in mild acidic condition once after radioisotope labeling. This requires that the Fmoc group should be removed and replaced with a Boc group.

ERGO has multiple advantages when it comes to studying oxidative stress in the brain, given the molecule is very water soluble, ideal for the clinical formulation whether it is for I.V. injection or for oral usage. But the paradox is that hydrophilic molecules cannot cross the blood-brain barrier (BBB). A unique feature of ERGO compared to other antioxidants is that its distribution to the brain parenchyma is independent of the BBB, but rather mediated by OCTN1 receptors. In clinical trials, either pure ERGO or mushrooms have been given to testing subjects for examining its effect on biomarkers of oxidative damage and inflammation. So far, all ERGO biodistribution and pharmacokinetics analysis rely on the indirect analysis of blood and urine samples.

The availability of the probe enables dynamic imaging to assess the biodistribution and kinetics of ERGO at any target of interest. Further, it also provides information for mapping OCTN1 receptors. For instance, the data in FIGS. 1.5A-B showed that within 5 minutes post I.V. injection, the compound started to present in the brain. At 10 minutes, ERGO is available almost in every region of the brain. The cortex and cerebellum had the highest regional uptake of the radiotracer compared to the hippocampus, striatum, and thalamus at every timepoint along with the TAC (FIG. 1.5B). This observation is consistent with a past study that showed ERGO was found in a larger quantity in the cerebellum than in the rest of the brain across different species, including mice, rats, guinea-pigs, cats, and sheep [46]. The presence and variable distribution of ERGO in the brain, with high concentrations in the central nervous system suggest the possibility of its role in the central regulatory function. In addition, without wishing to be bound by theory, based upon the observation of ERGO presence in the hippocampus of 5XFAD mouse it is believed that ERGO has implications in Alzheimer's disease.

The retention of the [¹¹C]ERGO probe depicted by strong signal in the brains of 5XFAD mice during PET scans (FIGS. 1.5A-B) was presumed due to the chronic neuroinflammation that occurs in Alzheimer's disease. As a stronger demonstration of the specificity of the probe, LPS-induced mice were used as a model of acute neuroinflammation. LPS is a molecular motif structurally similar amongst gram-negative bacteria that is recognized by the innate immune system and results in pro-inflammatory cytokine release mediated by toll-like receptor 4 (TLR-4). The present inventors have previously shown that injection of LPS (I.V.) resulted in severed neuroinflammation that breached the blood-brain barrier even at a nonlethal, low dose (<3 mg/Kg). Following observations of lethality at a dose greater than 3 mg/Kg combined with recent literature [51], LPS was injected into the peritoneum to prevent a robust inflammatory response. The 30-min PET scans (FIGS. 1.6A-C) suggested that ERGO was retained in the brain, likely acting as an adaptive antioxidant. The signal intensity of the LPS-treated mice was significantly higher than that found in control mice. It is also apparent that the specific distribution and retention of the probe is ROS-dependent, not because of the LPS-induced BBB opening, since pretreatment of LPS mice with Tempol, a general-purpose antioxidant known for scavenging a wide range of ROS and reactive nitrogen species, resulted in a remarkable reduction in the [¹¹C]ERGO signal across all the brain regions (FIGS. 1.6A-C).

Similar to the brain study, data from the whole-body biodistribution showed that 5 minutes post-injection, ERGO started to present in the peripheral tissues, as the levels in the blood reduced significantly (FIG. 1.7). Five minutes later, the compound is distributed mostly in every tissue. In this regard, it is noted that the level of ERGO is very high in the eyes and the small intestines. This in vivo observation is consistent with the past data, which also reported ERGO primarily concentrates in those tissues. Given the photo-oxidative process, the eyes are also susceptible to ROS and oxidative stress, and subsequent inflammatory conditions. Based on the data shown in FIGS. 1.6A-C (coronal and sagittal information), without wishing to be bound by theory, it is believed that this [¹¹C]ERGO probe could detect the inflammation (LPS-treated) and response (after treating with Tempol) to the treatment in the eyes.

Different from the in vivo PET imaging information, the biodistribution data showed that over the thirty minutes after administration, the ERGO continues to accumulate in most of the tissues, including the brain (FIG. 1.7). This discrepancy may be due to blood flow effects that are present only in the microPET data. Any signal due to ERGO presence in the blood is absent in the biodistribution data since perfusion of the vasculature was performed prior to measurement of the radioactivity in excised tissues.

### CONCLUSION

In summary, a new [¹¹C]ERGO PET radioligand has been synthesized to facilitate in vivo, non-invasive and real-time imaging of the biodistribution of ERGO. The development of a probe with an identical structure of ERGO is a significant advantage to our design. As such, all pharmacokinetics and biodistribution shown in this work are expected to reflect those of native compounds. Further, the availability of the PET radioligand enables the performance of longitudinal investigations in the same animals. Thus, differences between animals due to inter-individual variations can be controlled for. Overall, we anticipate that this probe will pave the way for the integration of molecular imaging with food biomarkers and biomedical research. The probe provides an emerging capability that will benefit the ERGO research community.

### REFERENCES FOR EXAMPLE 1

1. Tang R M Y, Cheah I K, Yew T S K, Halliwell B. Distribution and accumulation of dietary ergothioneine and its metabolites in mouse tissues. Sci Rep. 2018; 8:1601. doi:10.1038/s41598-018-20021-z.
2. Halliwell B, Cheah I K, Drum C L. Ergothioneine, an adaptive antioxidant for the protection of injured tissues? A hypothesis. Biochem Biophys Res Commun. 2016; 470:245-50. doi:10.1016/j.bbrc.2015.12.124.
3. Tanret C. Sur une base novelled retiree du seigle ergote, l'ergothioneine. Rend Acad Sci. 1909; 149:222-22.
4. Paul B D, Snyder S H. The unsual amino acid L-ergothioneine is a physiologic cytoprotectant. Cell Death Differ. 2010; 17:1134-40.
5. Motohashi N, Mori I, Sugiura Y. 13C-Nuclear Magnetic Resonance and Raman Spectroscopic Studies on Ionization and Mercury Complex of Ergothioneine. Chem Pharm Bull. 1976; 24:1737-41.
6. Beelman R B, Kalaras M D, Richie J P. Micronutrients and bioactive compounds in mushrooms. Nutrition Today. 2019; 54:The 16-22.
7. Dubost N J, Beelman R B, Peterson D, Royse D J. Identification and quantification of ergothioneine in cultivated mushrooms by liquid chromatochraphy-mass spectroscopy. Int J Med Mushrooms. 2006; 8:We 215-22.
8. Kalaras M D, Richie J P, Calcagnotto A, Beelman R B. Mushrooms: A rich source of the antioxidants ergothioneine and glutathione. Food Chem. 2017; 233:429-33. doi:10.1016/j.foodchem.2017.04.109.
9. Dubost N J, Ou B, Beelman R B. Quantification of polyphenols and ergothioneined in cultivated mushrooms and correlation to total antioxidant capacity. Food Chem. 2007; 105:727-35.
10. Dubost N J, Beelman R B, Royse D J. Influence of selected cultural factors and postharvest storage on ergothioneine content of common button mushroom Agaricus bisporus. Int J Med Mushrooms. 2007; 9:163-76.
11. Borodina I, Kenny L C, McCarthy C M, Paramasivan K, Pretorius E, Roberts T J, et al. The biology of ergothioneine, an antioxidant nutraceutical. Nutr Res Rev. 2020; 33:190-217. doi:10.1017/S0954422419000301.
12. Kerley R N, McCarthy C, Kell D B, Kenny L C. The potential therapeutic effects of ergothioneine in pre-eclampsia. Free Radic Biol Med. 2018; 117:145-57. doi:10.1016/j.freeradbiomed.2017.12.030.
13. Samuel P, Tsapekos M, de Pedro N, Liu A G, Casey Lippmeier J, Chen S. Ergothioneine Mitigates Telomere Shortening under Oxidative Stress Conditions. J Diet Suppl. 2020:1-14. doi:10.1080/19390211.2020.1854919.
14. Kumosani T A. L-ergothioneine level in red blood cells of healthy human males in the Western province of Saudi Arabia. Exp Mol Med. 2001; 33:20-2.
15. Nakamichi N, Nakayama K, Ishimoto T, Masuo Y, Wakayama T, Sekiguchi H, et al. Food-derived hydrophilic antioxidant ergothioneine is distributed to the brain and exerts antidepressant effect in mice. Brain Behay. 2016; 6:e00477. doi:10.1002/brb3.477.
16. Deiana M, Rosa A, Casu V, Piga R, Assunta Dessi M, Aruoma 01. L-ergothioneine modulates oxidative damage in the kidney and liver of rats in vivo: studies upon the profile of polyunsaturated fatty acids. Clin Nutr. 2004; 23:183-93. doi:10.1016/S0261-5614(03)00108-0.
17. Shires T K, Brummel M C, Pulido J S, Stegink L D. Ergothioneine distribution in bovine and porcine ocular tissues. Comp Biochem Physiol C Pharmacol Toxicol Endocrinol. 1997; 117:117-20. doi:10.1016/s0742-8413(96)00223-x.
18. Cheah I K, Tang R, Ye P, Yew T S, Lim K H, Halliwell B. Liver ergothioneine accumulation in a guinea pig model of non-alcoholic fatty liver disease. A possible mechanism of defence? Free Radic Res. 2016; 50:14-25. doi:10.3109/10715762.2015.1099642.
19. Lamhonwah A M, Tein I. Novel localization of OCTN1, an organic cation/carnitine transporter, to mammalian mitochondria. Biochem Biophys Res Commun. 2006; 345:1315-25. doi:10.1016/j.bbrc.2006.05.026.
20. Nakamichi N, Kato Y. Physiological Roles of Carnitine/Organic Cation Transporter OCTN1/SLC22A4 in Neural Cells. Biol Pharm Bull. 2017; 40:1146-52. doi:10.1248/bpb.b17-00099.
21. Nishida K, Takeuchi K, Hosoda A, Sugano S, Morisaki E, Ohishi A, et al. Ergothioneine ameliorates oxaliplatin-induced peripheral neuropathy in rats. Life Sci. 2018; 207:516-24. doi:10.1016/j.lfs.2018.07.006.
22. Song T Y, Lin H C, Chen C L, Wu J H, Liao J W, Hu M L. Ergothioneine and melatonin attenuate oxidative stress and protect against learning and memory deficits in C57BL/6J mice treated with D-galactose. Free Radic Res. 2014; 48:1049-60. doi:10.3109/10715762.2014.920954.
23. Yang N C, Lin H C, Wu J H, Ou H C, Chai Y C, Tseng C Y, et al. Ergothioneine protects against neuronal injury induced by beta-amyloid in mice. Food Chem Toxicol. 2012; 50:3902-11. doi:10.1016/j.fct.2012.08.021.
24. Aruoma O I, Spencer J P, Mahmood N. Protection against oxidative damage and cell death by the natural antioxidant ergothioneine. Food Chem Toxicol. 1999; 37:1043-53. doi:10.1016/s0278-6915(99)00098-8.
25. Colognato R, Laurenza I, Fontana I, Coppede F, Siciliano G, Coecke S, et al. Modulation of hydrogen peroxide-induced DNA damage, MAPKs activation and cell death in PC12 by ergothioneine. Clin Nutr. 2006; 25:135-45. doi:10.1016/j.clnu.2005.10.005.
26. Markova N G, Karaman-Jurukovska N, Dong K K, Damaghi N, Smiles K A, Yarosh D B. Skin cells and tissue are capable of using L-ergothioneine as an integral component of their antioxidant defense system. Free Radic Biol Med. 2009; 46:1168-76. doi:10.1016/j.freeradbiomed.2009.01.021.
27. Grundemann D, Harlfinger S, Golz S, Geerts A, Lazar A, Berkels R, et al. Discovery of the ergothioneine transporter. Proc Natl Acad Sci USA. 2005; 102:5256-61. doi:10.1073/pnas.0408624102.
28. Cheah I K, Feng L, Tang R M Y, Lim K H C, Halliwell B. Ergothioneine levels in an elderly population decrease with age and incidence of cognitive decline; a risk factor for neurodegeneration? Biochem Biophys Res Commun. 2016; 478:162-7. doi:10.1016/j.bbrc.2016.07.074.
29. Nakamichi N, Taguchi T, Hosotani H, Wakayama T, Shimizu T, Sugiura T, et al. Functional expression of carnitine/organic cation transporter OCTN1 in mouse brain neurons: possible involvement in neuronal differentiation. Neurochem Int. 2012; 61:1121-32. doi:10.1016/j.neuint.2012.08.004.
30. Shimizu T, Masuo Y, Takahashi S, Nakamichi N, Kato Y. Organic cation transporter Octn1-mediated uptake of food-derived antioxidant ergothioneine into infiltrating macrophages during intestinal inflammation in mice. Drug Metab Pharmacokinet. 2015; 30:231-9. doi:10.1016/j.dmpk.2015.02.003.
31. Taubert D, Jung N, Goeser T, Schomig E. Increased ergothioneine tissue concentrations in carriers of the Crohn's disease risk-associated 503F variant of the organic cation transporter OCTN1. Gut. 2009; 58:312-4. doi:10.1136/gut.2008.164418.
32. Tschirka J, Kreisor M, Betz J, Grundemann D. Substrate Selectivity Check of the Ergothioneine

Transporter. Drug Metab Dispos. 2018; 46:779-85. doi:10.1124/dmd.118.080440.
33. Moncaster J A, Walsh D T, Gentleman S M, Jen L S, Aruoma O I. Ergothioneine treatment protects neurons against N-methyl-D-aspartate excitotoxicity in an in vivo rat retinal model. Neurosci Lett. 2002; 328:55-9. doi:10.1016/50304-3940(02)00427-5.
34. Song T Y, Chen C L, Liao J W, Ou H C, Tsai M S. Ergothioneine protects against neuronal injury induced by cisplatin both in vitro and in vivo. Food Chem Toxicol. 2010; 48:3492-9. doi:10.1016/j.fct.2010.09.030.
35. Beelman R B, Kalaras M D, Phillips A T, Richie J P, Jr. Is ergothioneine a ‘longevity vitamin’ limited in the American diet? J Nutr Sci. 2020; 9:e52. doi:10.1017/jns.2020.44.
36. Morillon A C, Williamson R D, Baker P N, Kell D B, Kenny L C, English J A, et al. Effect of L-Ergothioneine on the metabolic plasma profile of the RUPP rat model of pre-eclampsia. PLoS One. 2020; 15:e0230977. doi:10.1371/journal.pone.0230977.
37. Williamson R D, McCarthy F P, Manna S, Groarke E, Kell D B, Kenny L C, et al. L-(+)-Ergothioneine Significantly Improves the Clinical Characteristics of Preeclampsia in the Reduced Uterine Perfusion Pressure Rat Model. Hypertension. 2020; 75:561-8. doi:10.1161/HYPERTENSIONAHA.119.13929.
38. Calvo M S, Mehrotra A, Beelman R B, Nadkarni G, Wang L, Cai W, et al. A Retrospective Study in Adults with Metabolic Syndrome: Diabetic Risk Factor Response to Daily Consumption of Agaricus bisporus (White Button Mushrooms). Plant Foods Hum Nutr. 2016; 71:245-51. doi:10.1007/s11130-016-0552-7.
39. Mehrotra A, Calvo M S, Beelman R B, Levy E, Siuty J, Kalaras M D, et al. Bioavailability of vitamin D2 from enriched mushrooms in prediabetic adults: a randomized controlled trial. Eur J Clin Nutr. 2014; 68:1154-60. doi:10.1038/ejcn.2014.157.
40. Butterfield D A, Halliwell B. Oxidative stress, dysfunctional glucose metabolism and Alzheimer disease. Nat Rev Neurosci. 2019; 20:148-60. doi:10.1038/s41583-019-0132-6.
41. Feng L, Cheah I K, Ng M M, Li J, Chan S M, Lim S L, et al. The Association between Mushroom Consumption and Mild Cognitive Impairment: A Community-Based Cross-Sectional Study in Singapore. J Alzheimers Dis. 2019; 68:197-203. doi:10.3233/JAD-180959.
42. Yatin S M, Varadarajan S, Link C D, Butterfield D A. In vitro and in vivo oxidative stress associated with Alzheimer's amyloid beta-peptide (1-42). Neurobiol Aging. 1999; 20:325-30; discussion 39-42. doi:10.1016/s0197-4580(99)00056-1.
43. Cheah I K, Tang R M, Yew T S, Lim K H, Halliwell B. Administration of Pure Ergothioneine to Healthy Human Subjects: Uptake, Metabolism, and Effects on Biomarkers of Oxidative Damage and Inflammation. Antioxid Redox Signal. 2017; 26:193-206. doi:10.1089/ars.2016.6778.
44. Xu T, Beelman R B, Lambert J D. The cancer preventive effects of edible mushrooms. Anticancer agents Med Chem. 2012; 12:1255-63.
45. Zhang S, Sugawara Y, Chen S, Beelman R B, Tsuduki T, Tomata Y, et al. Mushroom consumption and incident risk of prostate cancer in Japan: A pooled analysis of the Miyagi Cohort Study and the Ohsaki Cohort Study. Int J Cancer. 2020; 146:2712-20. doi:10.1002/ijc.32591.
46. Crossland J, Mitchell J, Woodruff G N. The presence of ergothioneine in the central nervous system and its probable identity with the cerebellar factor. J Physiol. 1966; 182:427-38. doi:10.1113/jphysiol.1966.sp007830.
47. Cheah I K, Ng L T, Ng L F, Lam V Y, Gruber J, Huang C Y W, et al. Inhibition of amyloid-induced toxicity by ergothioneine in a transgenic Caenorhabditis elegans model. FEBS Lett. 2019; 593:2139-50. doi:10.1002/1873-3468.13497.
48. Kim S J, Russell A E, Wang W, Gemoets D E, Sarkar S N, Simpkins J W, et al. miR-146a Dysregulates Energy Metabolism During Neuroinflammation. J Neuroimmune Pharmacol. 2021. doi:10.1007/s11481-021-09999-y.
49. Awasthi S. Toll-like receptor-4 modulation for cancer immunotherapy. Front Immunol. 2014; 5:328. doi:10.3389/fimmu.2014.00328.
50. Barton S M, Janve V A, McClure R, Anderson A, Matsubara J A, Gore J C, et al. Lipopolysaccharide induced opening of the blood brain barrier on aging 5XFAD mouse model. J Alzheimer Disease. 2019; 67:503-13.
51. Hou C, Hsieh C J, Li S, Lee H, Graham T J, Xu K, et al. Development of a Positron Emission Tomography Radiotracer for Imaging Elevated Levels of Superoxide in Neuroinflammation. ACS Chem Neurosci. 2018; 9:578-86. doi:10.1021/acschemneuro.7b00385.
52. Francischetti I M, Gordon E, Bizzarro B, Gera N, Andrade B B, Oliveira F, et al. Tempol, an intracellular antioxidant, inhibits tissue factor expression, attenuates dendritic cell function, and is partially protective in a murine model of cerebral malaria. PLoS One. 2014; 9:e87140. doi:10.1371/journal.pone.0087140.
53. Babizhayev M A, Yegorov Y E. Biomarkers of oxidative stress and cataract. Novel drug delivery therapeutic strategies targeting telomere reduction and the expression of telomerase activity in the lens epithelial cells with N-acetylcarnosine lubricant eye drops: anti-cataract which helps to prevent and treat cataracts in the eyes of dogs and other animals. Curr Drug Deliv. 2014; 11:24-61. doi:10.2174/15672018113106660062.
54. Herfert K, Mannheim J G, Kuebler L, Marciano S, Amend M, Parl C, et al. Quantitative Rodent Brain Receptor Imaging. Mol Imaging Biol. 2020; 22:223-44. doi:10.1007/s11307-019-01368-9.
55. McClure R, Ong H, Janve V, Barton S, Zhu M, Li B, et al. Aerosol Delivery of Curcumin Reduced Amyloid-beta Deposition and Improved Cognitive Performance in a Transgenic Model of Alzheimer's Disease. J Alzheimers Dis. 2017; 55:797-811. doi:10.3233/JAD-160289.
56. McClure R, Yanagisawa D, Stec D, Abdollahian D, Koktysh D, Xhillari D, et al. Inhalable curcumin: offering the potential for translation to imaging and treatment of Alzheimer's disease. J Alzheimers Dis. 2015; 44:283-95. doi:V268KVP65021QJ19 [pii] 10.3233/JAD-140798.
57. McClure R A, Chumbley C W, Reyzer M L, Wilson K, Caprioli R M, Gore J C, et al. Identification of promethazine as an amyloid-binding molecule using a fluorescence high-throughput assay and MALDI imaging mass spectrometry. Neurolmage: Clinical. 2013; 2:620-9.

### Example 2

L-ergothioneine (ERGO) is a potent antioxidant synthesized by actinomycetes, cyanobacteria, methylbacteria, and some fungi, like mushrooms [1]. It has been reported that ERGO can prevent cell and tissue damage, a key contributor to aging, by protecting against free radicals and oxidative stress [2-4]. The chemical structure of ERGO is similar to that of histidine but with the presence of a thione group on the imidazole ring [5]. It is reported that ERGO protects against cytotoxicity by scavenging singlet oxygen, hydroxyl radicals [6-8], hypochlorous acid (HOCl), peroxyl radicals [8, 9], and acting as an inhibitor of iron or copper ion-dependent generation of hydroxyl radicals from hydrogen peroxide [6]. This protection is derived from its conspicuous affinity for metal cations, such as Fe and Cu, permitting capture and neutralization of associated radicals [10]. Thus, ERGO is considered a potent antioxidant with potential therapeutic implications. In particular, this antioxidant might play essential roles in the central nervous system [11-13], given the substantial research data indicating that ERGO can be distributed to the brain via the OCTN1 transporter [10].

Our group recently reported the synthesis of an [¹¹C]ERGO PET radioligand to facilitate in vivo imaging of the biodistribution and pharmacokinetics using preclinical mouse models [5]. The work also demonstrated the specificity of the probe for the detection of LPS-induced inflammation and oxidative stress. In the previous synthesis, the carboxylic group of histidine was protected as a t-butyl ester for the synthesis of the precursor. After labeling with a [¹¹C]CH₃ positron emitter, all of the acidic-labile protecting groups, including the t-butyl ester, were cleaved by treating with hydrochloric acid at 80° C. The advantage of using t-butyl ester is its sensitivity to an acidic condition, albeit with sufficient stability to survive very mild acidic conditions at low temperature. This feature serves best when robust deprotection conditions are required. The t-butyl ester's strength, however, is also its shortcoming. Its sensitivity in an acidic condition made reaction workup harder than usual, and thus, low reaction yield is inevitable.

The issue mentioned above is further compounded by the reproducibility problem pertaining to the presence of the t-butyl ester. To overcome these drawbacks, we switched the t-butyl ester group to a more stable methyl ester counterpart, although this new chemistry needs one additional step during radioisotope labeling. The reproducibility of this design is the real impetus behind this work. This article describes an improved synthesis with an overall yield of 24% starting from the methyl ester to the precursor, compared to 14% as reported in the past [5].

Given the apparent implications of reactive oxygen species (ROS) in mitochondrial dysfunction related to the pathogenesis of AD [14], we used the resultant [¹¹C]ERGO PET radioligand to test the incipient hypothesis that oxidative stress could potentially serve as a biomarker for AD [15-18]. Here, we present the data demonstrating that the probe can detect oxidative stress in the brains of a common mouse model for Alzheimer's disease (AD) neuropathology 5XFAD mice. The in vivo PET imaging showed the probe was retained in the brain of 5XFAD mice with a higher concentration than wild-type (WT) counterparts. As expected, this is due to the trapping of the probe, given its high affinity for ROS products and oxidized metals associated with AD [19-23]. Overall, the [¹¹C]ERGO PET radioligand enables us to observe, for the first time, the dynamics of this antioxidant in action noninvasively in a live subject.

### Results

Development of a precursor. The carboxylic group of protected histidine 1 was capped as a methyl ester by a routine procedure using 1-ethyl-3-(3-dimethylaminopropyl)carbodiimide (EDC) with a trace 4-dimethylaminopyridine (DMAP) as a catalyst in methanolic dichloromethane (FIG. 2.1). Since three active groups were orthogonally protected with a different group, the tert-butyloxycarbonyl (Boc) protecting group could be cleaved independently from the others using trifluoroacetic acid (TFA) to provide a high reaction yield. Next, reductive methylation of a primary amine with aldehyde and sodium cyanoborohydride to produce intermediate 4 with good yield. The incorporation of the thioketone into the imidazole ring and protection of the active functional groups in imidazole ring with Boc groups were performed as described in the past to provide precursor 6 [5].

Radiosynthesis of [¹¹C]ERGO PET radioligand. Direct methylation of precursor 6 was achieved using [¹¹C]MeOTf in acetonitrile (ACN) (FIG. 2.2). The Boc protecting groups were removed by treatment with TFA, followed by ester hydrolysis in the presence of 5.0 M aqueous sodium hydroxide at 70° C. for 4 min. The product was produced with 55±10% radiochemical purity (decay corrected, n=5) confirmed by TLC and HPLC, and a molar activity of 450±200 TBq/mmol. The identity of [¹¹C]ERGO was confirmed by comparison with non-radioactive reference compounds. The retention time of [¹¹C]ERGO was 7.6 min, while the reference compound was 7.3 min.

Dynamic uptake and retention of [¹¹C]ERGO radioligand. Two cohorts of age-matched mice (10-month-old, n=4, each), including 5XFAD and WT animals, were injected with a consistent amount of 14.8 MBq of [¹¹C]ERGO in 0.1 mL via the tail vein prior to acquiring dynamic imaging. Our previous PET imaging data indicated that ERGO started to present in the brain 5 min post-injection, and it continued to accumulate until 30 min before saturation [5]. In this work, we decided to run 20-min dynamic scans to test, as a proof-of-concept, whether the affinity of ERGO for radicals and/or metals, which are integrated in or very closely associated with Abeta aggregates [24-29], would trap the probe. As shown in a representative set of dynamic scans along with the corresponding time-activity curves (TAC) (FIG. 2.3), [¹¹C]ERGO radioligand accumulated significantly higher in every subregions of 5XFAD brain (FIG. 2.3A, 2.3B, 2.3C, 2.3D) compared to WT mice (FIG. 2.3E, 2.3F, 2.3G, 2.3H). The mean percent of ID/g in the brains of 5XFAD mice (n=4) were 0.81±0.09, 0.72±0.09, 0.72±0.10, 0.71±0.12, 0.72±0.08, 0.83±0.10 for cortex, hippocampus, striatum, thalamus, cerebellum and whole brain, respectively, whereas for WT mice (n=4), the values were 0.52±0.13, 0.47±0.18, 0.47±0.15, 0.42±0.18, 0.51±0.13, 0.55±0.14. Overall, the uptake in 5XFAD brains was significantly (p<0.05) higher than that of WT control. 5XFAD brains are associated with high levels of GFAP and IBA1 expression. After PET/CT imaging, animals underwent through the cardiac perfusion process, and the brains were embedded in OCT solution for micro-sectioning. FIG. 2.4 shows the representative photomicrographs of brain slices (5-8-μm thickness) stained for astrocyte (green) and microglia (red) protein markers in DAPI (blue) positive cells. The hippocampal regions of 5XFAD mice, including the dentate gyrus (DG), Cornu ammonis (CA) and the surrounding regions are populated with astrocytes, which were detected and quantified by immunostaining using anti-GFAP antibody. In contrast, very low amounts of reactive astrocytes were observed in the brains of WT mice. Quantitatively, about 2-fold higher levels of this inflammatory marker is found in 5XFAD versus control mice (*p<0.05). In a similar observation, 5XFAD mouse brains have greater numbers of IBA1-positive activated microglia (arrows) compared to WT counterparts. The level of activated microglia in WT mice is nearly negligible. Detailed pixel count and quantitative analysis show that the number of activated microglia cells in the hippocampus of 5XFAD mice over 20-fold (**p<0.01).

### Experimental Methods

### General

All solvents and chemicals were purchased from common vendors at reagent grade and were used without further purifications. ¹H- and ¹³C-NMR spectra were obtained using a Bruker 600-MHz NMR spectrometer equipped with cryogenic radio frequency probe.

Compound 2. To a stirring solution of 1 (535 mg, 1.12 mmol) in CH₂Cl₂ (10 mL) and methanol (5 mL) at 0° C. was added with EDC-HCl (257 mg, 1.3 mmol), and DMAP (25 mg, 0.22 mmol. This solution was allowed to warm up to room temperature (r.t.) and stirred overnight. The reaction was diluted with H₂O and CH₂Cl₂. The product was extracted 3× with CH₂Cl₂. The organic layers were combined, washed with 1M HCl, saturated sodium bicarbonate, brine, dried over Na₂SO₄, filtered, and concentrated under reduced pressure. Purified by flash chromatography (0-50% CH₂Cl₂/(20% MeOH/CH₂Cl₂)), (241 mg, 45% yield). ¹H CDCl₃ (600.13 mHz): 7.97 (d, J=0.6 Hz, 1H); 7.80 (d, J=7.2 Hz, 2H); 7.57 (d, J=7.8 Hz, 2H); 7.44, (t, J=7.2 Hz, 2H); 7.35 (t, J=7.8 Hz, 2H); 7.16 (s, 1H); 5.66 (d, J=7.8 Hz, 1H); 4.72 (d, J₁=6.6 Hz, 2H); 4.60 (m, 1H); 4.35 (t, J=6.6 Hz, 1H); 3.72 (s, 3H); 3.09 (dd, J₁=15.0 Hz, J₂=5.4 Hz, 1H); 3.04 (dd, J₁=15.0 Hz, J₂=4.8 Hz, 1H); 1.44 (s, 9H). ¹³C CDCl₃ (150.9 mHz): 172.3, 155.6, 148.5, 142.8, 141.5, 139.5, 137.0, 128.4, 127.6, 124.9, 120.5, 114.6, 80.0, 69.9, 53.2, 52.5, 46.7, 30.4, 28.5.

Compound 3. To a stirring solution of 2 (241 mg, 0.50 mmol) in CH₂Cl₂ (20 mL) at −78° C. was added TFA (2 mL) dropwise. This solution was allowed to warm up to 0° C. and stirred until completion. The solution was then neutralized with sodium bicarbonate and diluted with H₂O and CH₂Cl₂. The product was extracted 3× with CH₂Cl₂. The organic layers were combined, washed with brine, dried over Na₂SO₄, filtered, and concentrated under reduced pressure. The product was purified by flash chromatography (0-50% CH₂Cl₂/(20% MeOH/CH₂Cl₂)), (172 mg, 88% yield). ¹H CDCl₃ (600.13 mHz): 8.24 (s, 1H); 7.79 (d, J=7.2 Hz, 2H); 7.54 (d, J=7.8 Hz, 2H); 7.43, (t, J=7.2 Hz, 2H); 7.40, (s, 1H); 7.33 (t, J=7.2 Hz, 2H); 4.78 (dd, J₁=6.6 Hz, J₂=1.8 Hz, 2H); 4.43 (br.s, 1H); 4.35 (t, J=6.6 Hz, 1H); 3.79 (s, 3H); 3.38 (d, J=13.8 Hz, 1H); 3.30 (dd, J₁=15.6 Hz, J₂=6.6 Hz, 1H). ¹³C CDCl₃ (150.9 mHz): 168.4, 147.3, 142.4, 141.5, 136.9, 134.8, 128.5, 127.6, 124.8, 120.5, 116.6, 71.1, 53.8, 52.9, 46.6, 26.4.

Compound 4. To a stirring solution of 3 (172 mg, 0.44 mmol) in MeOH (10 mL) was added NaBH₃CN (63 mg, 1.0 mmol) and CH₂O (37% in H₂0, 90 mg, 3.0 mmol). The reaction was capped and stirred overnight. The resulting solution was then concentrated via rotovap. Purified by reverse phase flash chromatography (0-50% H₂O/ACN), (68 mg, 79% yield). The FMOC group was removed in this step through interaction with the NaBH₃CN. ¹H CDCl₃ (400.13 mHz): 7.47 (s, 1H); 6.75 (s, 1H); 3.61 (s, 3H); 3.48 (t, J=7.6 Hz, 1H); 3.01 (dd, J₁=14.8 Hz, J₂=8.0 Hz, 1H); 2.88 (dd, J₁=14.8 Hz, J₂=6.8 Hz, 1H); 2.33 (s, 6H). ¹³C CDCl₃ (100.6 mHz): 172.0, 134.8, 133.1, 118.5, 67.6, 51.3, 41.7, 26.5.

Compound 5. To a stirring solution of 4 (68 mg, 0.34 mmol) in H₂O (2 mL) and diethyl ether (2 mL) was added NaHCO₃ (211 mg, 2.5 mmol), and phenyl chlorothionoformate (158 mg, 0.42 mmol) and stirred overnight. Reaction was diluted with H₂O and diethyl ether. The product was extracted 3× with diethyl ether. The organic layers were combined, washed with brine, dried over Na₂SO₄, filtered, and concentrated under reduced pressure. The resulting oil was then redissolved in MeOH (5 mL) and triethylamine (174 μL) was added. This solution was stirred overnight. The resulting solution was then concentrated under reduced pressure. The final product was purified by flash chromatography (0-50% CH₂Cl₂/(20% MeOH/CH₂Cl₂)), (50 mg, 64% yield).

Compound 6. To a stirring solution of 5 (50 mg, 0.218 mmol) in CH₂Cl₂ (6 mL) was added: Boc anhydride (190 mg, 0.873 mmol), and DIPEA (113 mg, 0.873 mmol) was heated to 37° C. overnight. The reaction was cooled to r.t. and diluted with CH₂Cl₂ and H₂O. The product was extracted 3× with CH₂Cl₂. The organic layers were combined, washed with brine, dried over Na₂SO₄, filtered, and concentrated under reduced pressure. Purified by flash chromatography (0-50% CH₂Cl₂/(20% MeOH/CH₂Cl₂)), (50 mg, 54% yield). ¹H CDCl₃ (400.13 mHz): 7.36 (s, 1H); 3.65 (s, 3H); 3.63 (m, 1H); 2.88 (dd, J₁=14.4 Hz, J₂=8.4 Hz, 1H); 2.86 (dd, J₁=14.4 Hz, J₂=6.0 Hz, 1H); 2.35 (s, 6H); 1.58 (s, 9H), 1.46 (s, 9H). ¹³C CDCl₃ (100.6 mHz): 171.9, 165.3, 146.7, 139.7, 135.2, 119.8, 86.8, 85.9, 66.7, 51.3, 41.8, 29.8, 28.2, 27.9.

### Radiosynthesis of [¹¹C]ERGO PET Radioligand

The [¹¹C]ERGO radioligand was prepared using the GE Healthcare Tracerlab FXC-Pro, a commercially supplied reaction platform, modified to directly connect V7_{nc} to V11ₙₒ. The [¹¹C]CO₂ is made by irradiating a target filled with nitrogen and 1% oxygen gas with protons. The [¹¹C]CO₂ is then trapped on nickel Shimalite with molecular sieves at room temperature. The [¹¹C]CO₂ is then converted to [¹¹C]CH₄ by heating the trapped [¹¹C]CO₂ to 400° C. in the presence of hydrogen gas. The [¹¹C]CH₄ is then released from the nickel Shimalite at 400° C. and isolated on molecular sieves at −75° C. The [¹¹C]CH₄ is then converted to [¹¹C]MeI via a recirculation through gaseous iodine at ˜720° C., with the [¹¹C]MeI being trapped on Porapak N with each cycle. The [¹¹C]MeI is then released from the Porapak N by heating with a gentle flow of helium that is passed through an AgOTf impregnated column at ˜200° C. to convert the [¹¹C]MeI to [¹¹C]MeOTf; this [¹¹C]MeOTf is bubbled into a solution of precursor in 250 μI acetonitrile at −10° C. After the activity transfer is complete the reaction mixture was heated to 80° C. for 2 minutes. At this time trifluoroacetic acid (250 μl) was added, the reaction mixture heated at 70° C. for 3 minutes, 5M of aqueous NaOH (1 mL) added and heated at 70° C. for 4 minutes. The reaction mixture was cooled to room temperature, passed through an ion-retardation resin (Ag11-A8, 3 g) into the product vial and the resin was rinsed with water (5 mL) into the final product vial.

We labeled the precursor 6 with [¹¹C]CH₃OTf using a commercial automated radiosynthesis module (GE TRACERlab FXc Pro). [¹¹C]CO₂ was converted to [¹¹C]MeOTf using the standard reaction conditions, reacted with precursor 6 at 80° C. to give the protected intermediate. The Boc-groups were removed under acidic conditions followed by the use of 5M aqueous sodium hydroxide both to neutralize the TFA, and for the saponification of the methyl ester to provide an [¹¹C]ERGO radioligand. The [¹¹C]ERGO was passed through an ion-retardation resin, the pH was adjusted with aqueous HCl before use.

### Animals

All animal experiments performed complied with institutional guidelines and were conducted according to the protocol approved by the Vanderbilt Institutional Animal Care and Use Committee. The 5XFAD mice were maintained at Vanderbilt University under standard conditions, in a 12-h light/dark cycle and with free access to food and water. The 5XFAD mice over express both mutant human APP and PS1 genes and it correlates with high APP levels correlating with high burden and accelerated accumulation of β-amyloid (Abeta). A colony of 5XFAD transgenic mice obtained from Jackson Laboratories was maintained by crossing 5XFAD mice with a wild-type (WT) C57BL/6J strain. The 5XFAD mice were maintained as heterozygous.

Dynamic PET imaging. The dynamic acquisition was divided into twelve 5 sec frames, four 60 sec frames, five 120 sec frames, three 5 min frames, and six 10 min scans. The data from all possible lines of response (LOR) were saved in the list mode raw data format. The raw data were then binned into 3D sinograms with a span of 3 and ring difference of 47. The images were reconstructed into transaxial slices (128×128×159) with voxel sizes of 0.0815×0.0815×0.0796 cm³, using the MAP algorithm with 16 subsets, 4 iterations, and a beta of 0.0468. For anatomical co-registration, immediately following the PET scans, the mice received a CT scan in a NanoSPECT/CT (Mediso, Wash. D.C.) at an X-ray beam intensity of 90 mAs and x-ray peak voltage of 45 kVp. The CT images were reconstructed into 170×170×186 voxels at a voxel size of 0.4×0.4×0.4 mm³. The PET/CT images were uploaded into Amide software (www.sourceforge.com), co-registered to an MRI template made in-house, and volumetric regions-of-interest were drawn around the cortex, hippocampus, striatum, thalamus, and cerebellum in addition to the whole brain. The PET images were normalized to the injected dose, and the time-activity-curves (TACs) of the mean activity within the ROIs were estimated for the entire duration of the scans.

Cardiac perfusion procedure and tissue collection. A sharp incision into the abdomen of the anesthetized mouse was made, followed by a longitudinal cut with a scalpel to open the thoracic cavity, which then was stabilized with a retractor. Perfusion began with a 20-gauge syringe containing ice cold PBS (30 ml, pH 7.4) in the left ventricle while the atrium was snipped off. This was followed by injection of paraformaldehyde (PFA) solution (4%). Once the perfusion was completed, the animals were decapitated, and the brains were quickly removed and fixed in PFA overnight at 4° C. followed by sucrose precipitation (30%) overnight at 4° C. The brains were then embedded in Cryo-OCT compound (Fisher Scientific) before sectioning.

Ex vivo brain cross-sections processed for GFAP and IBA1 Immunohistochemistry. The embedded brains in OCT were cut into coronal sections (10 μm) using a Tissue-Tek cryostat and mounted onto charged glass slides. Prior to staining, slides were washed with PBS (3×, 5,5, and 10 min); then, they were treated with blocking buffer (5% normal goat serum, 0.2% Triton X-100, 0.5% bovine albumin in PBS) for 1 h at room temperature. The treated sections were then incubated overnight at 4° C. with primary anti-GFAP antibody (1:100 dilution, Biolegend San Diego, Calif., USA) or anti-IBA1 antibody (1:250 dilution, FUJIFILM Wako Chemicals U.S.A Corporation Richmond, Va., USA). Slides were washed with PBS (3×) for 10 min each, the sections stained for GFAP were subsequently incubated with secondary antibody goat anti-mouse Alexa Fluor 488 (1:200 dilution, Thermo Fisher Scientific, Carlsbad, Calif., USA). Sections stained for IBA1 were incubated with secondary antibody goat anti-rabbit Alexa Fluor 680 (1:200 dilution, Thermo Fisher Scientific, Carlsbad, Calif., USA) for 30 min at room temperature. The sections were then washed with PBS twice for 10 minutes and once for 30 minutes, and cover slipped with an antifade mounting medium with DAPI (Vector Laboratories, Burlingame, Calif.) before observation under a fluorescence microscope.

Statistical analysis. Data were converted to 8-bit grayscale and thresholding using Otsu's method; the pixels were quantified using ImageJ software (version 1.53). The data were then imported to GraphPad software (GraphPad Prism version 9.2.0 for Mac) for statistical analysis. The results are presented as mean±SEM. Differences were analyzed with student's t-test, and the results were considered significant at p values<0.05. For the IBA1 data, since the pixels are small due low expression, the RGB channels were separated, and the red channel, which indicates IBA1-positive signal were thresholded and analyzed as described.

### Discussion

ERGO possesses a unique chemical structure, which contains multiple functional groups. Thus, extensive protection and deprotection maneuvers are necessary to generate a precursor for [¹¹C]CH₃ labeling. In that case, many potential combinations of protecting groups have been explored to identify the most compatible deployment of these groups and their order. After several attempts, we reported a successful sequential order to reach to the dimethyl amine precursor 6. Basically, to set the stage for the crucial incorporation of the thiol group onto imidazole, the amino and carboxyl groups of histidine were blocked with acidic labile moieties, while the imidazole amine was protected with an Fmoc group. We previously reported that the amine-protecting Boc groups could be removed using TFA in the presence the t-butyl ester at −78° C. [5]. This reaction confers good yield, albeit only with stringent operations and attention to detail. And thus, the reproducibility is moderate in this case, which should come as no surprise since both the Boc and t-butyl ester share a similar acid-catalyzed hydrolysis mechanism (FIG. 2.5). In essence, both processes yield t-Bu⁺ adduct after C—O cleavage, followed by the formation of a more stable Me₂C═CH₂ moiety. In the case of the Boc group, the formation of t-Bu⁺ occurs with the presence of the carbamic acid, which accompanied by the release of CO₂ and the desired free amine. Thus, changing to other esters would be alleviate this problem. While methyl ester could be simply achieved through Fischer esterification by treating carboxylic 1 with methanol in the presence of acid. However, this will affect the Boc group, and thus we opted to use Steglich esterification using EDC and DMAP as a catalyst.

When the carboxylic group was protected as a methyl ester, the stability of this ester contributes to better overall reaction yield, 24% versus 14% in the case of tert-butyl ester starting from compounds 2 to 6. But that is only a part of the motivation for this development. We found that this new chemistry is more reliable and reproducible, a main feature necessary for any radiopharmaceutical development. It is certain that this improved and robust chemistry will foster more translational value of the probe for in vivo imaging applications.

We have also demonstrated the specificity of the probe for in vivo detection of oxidative stress using an acute inflammatory LPS mouse model [5]. To further understand the implications of oxidative stress in the context of Abeta cascade mechanism, we want to assess whether this probe could report specific activity relevant to a disease that manifests chronic presentation of an array of ROS-induced inflammation like AD [30-34]. As shown in FIG. 2.3, the probe was distributed to the brain and its retention in 5XFAD is more significant than that of control animals, in all brain subregions. The data suggest that Abeta-associated free radical oxidative stress might trap the probe, and thus its accumulation in the brain was reported in the PET images with enhanced spatial and temporal resolution compared to WT counterparts. Furthermore, the existence of metals, such as Zn and Cu, which potentiate AD by participating in the Abeta aggregation by generation of ROS, might offer the ob In essence, the detection of the probe retention via noninvasive PET imaging alludes to this being visualization of the probe in action.

In corroboration with the PET data, the immunohistochemistry staining of the brain sections showed that the hippocampus of 5XFAD mice exhibit higher expression levels of activated astrocytes and microglia compared to WT mice. The background level of GFAP-positive astrocytes in WT mice was low, but it could be detected; while IBA1-positive microglia (resting state) in WT mice were barely detectable at high resolution (data not shown). The upregulation of activated microglia and astrocytes support the hypothesis that neuroinflammation could be one of the mechanisms by which AD pathology, including Abeta, leads to neuronal death and dysfunction [35]. Particularly, the pro-inflammatory GFAP marker of the reactive astrocytes, those expression has been linked to Abeta plaque load [36-38]. Furthermore, accumulated data in the past showed that GFAP could serve as an early marker associated with brain Abeta pathology [39]. The detection of oxidative stress using the [¹¹C]ERGO radioligand confirms a past report, which showed that the formation of Abeta plaques trigger a proinflammatory response from microglia and astrocytes [40]. Taken together the data obtained from this work suggest that neuroinflammation reflected in activated microglia and astrocytes in this Abeta accumulating model may trap the [¹¹C]ERGO radioligand contributing to the enhanced PET signal in the 5XFAD mice.

In summary, we report an improved synthesis for the robust and reliable production of [¹¹C]ERGO PET radioligand. Further, the in vivo imaging of an AD phenotype using this probe provides a proof-of-principle to demonstrate for the first time that the neuroinflammation associated with AD could be detected non-invasively, and thereby establishes a novel platform for additional work in the future.

### REFERENCES FOR EXAMPLE 2

[1] Cumming B M, Chinta K C, Reddy V P, Steyn A J C (2018) Role of Ergothioneine in Microbial Physiology and Pathogenesis. Antioxid Redox Signal 28, 431-444.
[2] Borodina I, Kenny L C, McCarthy C M, Paramasivan K, Pretorius E, Roberts T J, van der Hoek S A, Kell D B (2020) The biology of ergothioneine, an antioxidant nutraceutical. Nutr Res Rev 33, 190-217.
[3] Kerley R N, McCarthy C, Kell D B, Kenny L C (2018) The potential therapeutic effects of ergothioneine in pre-eclampsia. Free Radic Biol Med 117, 145-157.
[4] Samuel P, Tsapekos M, de Pedro N, Liu A G, Casey Lippmeier J, Chen S (2020) Ergothioneine Mitigates Telomere Shortening under Oxidative Stress Conditions. J Diet Suppl, 1-14.
[5] Behof W J, Whitmore C A, Haynes J R, Rosenberg A J, Tantawy M N, Peterson T E, Harrison F E, Beelman R B, Pham W (2021) A novel antioxidant ergothioneine PET radioligand for in vivo imaging applications. Scientific Reports 11, 18450.
[6] Akanmu D, Cecchini R, Aruoma 01, Halliwell B (1991) The antioxidant action of L-Ergo. Arch Biochem Biophys 288, 10-16.
[7] Motohashi N, Mori 1 (1986) Thiol-induced hydroxyl radical formation and scavenger effect of thiocarbamides on hydroxyl radicals. J Inorg Biochem 26, 205-212.
[8] Asmus K D, Bensassion R V, Bernier J L, Houssin R, Land E J (1996) One electron oxidation of L-Ergo and analogues investigated by pulsed radiolysis: redox reactions involving L-Ergo and vitamin C. Biochem J 315, 625-629.
[9] Aruoma 01, Whiteman M, England T G, Halliwell B (1997) Antioxidant action of ergothioneine: assessment of its ability to scavenge peroxynitrite. Biochem Biophys Res Commun 231, 389-391.
[10] Grundemann D, Harlfinger S, Golz S, Geerts A, Lazar A, Berkels R, Jung N, Rubbert A, Schomig E (2005) Discovery of the ergothioneine transporter. Proc Natl Acad Sci USA 102, 5256-5261.
[11] Crossland J, Mitchell J, Woodruff G N (1966) The presence of ergothioneine in the central nervous system and its probable identity with the cerebellar factor. J Physiol 182, 427-438.
[12] Nakamichi N, Nakayama K, Ishimoto T, Masuo Y, Wakayama T, Sekiguchi H, Sutoh K, Usumi K, Iseki S, Kato Y (2016) Food-derived hydrophilic antioxidant ergothioneine is distributed to the brain and exerts antidepressant effect in mice. Brain Behav 6, e00477.
[13] Teruya T, Chen Y J, Kondoh H, Fukuji Y, Yanagida M (2021) Whole-blood metabolomics of dementia patients reveal classes of disease-linked metabolites. Proc Natl Acad Sci USA 118.
[14] Benzi G, Moretti A (1995) Are reactive oxygen species involved in Alzheimer's disease? Neurobiol Aging 16, 661-674.
[15] Abramov A Y, Canevari L, Duchen M R (2004) Beta-amyloid peptides induce mitochondrial dysfunction and oxidative stress in astrocytes and death of neurons through activation of NADPH oxidase. J Neurosci 24, 565-575.
[16] Bermejo P, Martin-Aragon S, Benedi J, Susin C, Felici E, Gil P, Ribera J M, Villar A M (2008) Peripheral levels of glutathione and protein oxidation as markers in the development of Alzheimer's disease from Mild Cognitive Impairment. Free Radic Res 42, 162-170.
[17] Casado A, Encarnacion Lopez-Fernandez M, Concepcion Casado M, de La Torre R (2008) Lipid peroxidation and antioxidant enzyme activities in vascular and Alzheimer dementias. Neurochem Res 33, 450-458.
[18] Govoni S, Lanni C, Racchi M (2001) Advances in understanding the pathogenetic mechanisms of Alzheimer's disease. Funct Neurol 16, 17-30.
[19] Atwood C S, Huang X, Moir R D, Tanzi R E, Bush A I (1999) Role of free radicals and metal ions in the pathogenesis of Alzheimer's disease. Met Ions Biol Syst 36, 309-364.
[20] Atwood C S, Scarpa R C, Huang X, Moir R D, Jones W D, Fairlie D P, Tanzi R E, Bush A I (2000) Characterization of copper interactions with alzheimer amyloid beta peptides: identification of an attomolar-affinity copper binding site on amyloid beta1-42. J Neurochem 75, 1219-1233.
[21] Bush A I (2000) Metals and neuroscience. Curr Opin Chem Biol 4, 184-191.
[22] Bush A I (2003) The metallobiology of Alzheimer's disease. Trends Neurosci 26, 207-214.
[23] Huang X, Cuajungco M P, Atwood C S, Hartshorn M A, Tyndall J D, Hanson G R, Stokes K C, Leopold M, Multhaup G, Goldstein L E, Scarpa R C, Saunders A J, Lim J, Moir R D, Glabe C, Bowden E F, Masters C L, Fairlie D P, Tanzi R E, Bush A I (1999) Cu(II) potentiation of alzheimer abeta neurotoxicity. Correlation with cell-free hydrogen peroxide production and metal reduction. J Biol Chem 274, 37111-37116.
[24] Butterfield D A (1997) Beta-Amyloid-associated free radical oxidative stress and neurotoxicity: implicaitons for Alzheimer's disease. Chem Res Toxicol 10, 495-506.
[25] Butterfield D A, Boyd-Kimball D (2020) Mitochondrial Oxidative and Nitrosative Stress and Alzheimer Disease. Antioxidants (Basel) 9.
[26] Butterfield D A, Halliwell B (2019) Oxidative stress, dysfunctional glucose metabolism and Alzheimer disease. Nat Rev Neurosci 20, 148-160.
[27] Cheignon C, Tomas M, Bonnefont-Rousselot D, Faller P, Hureau C, Collin F (2018) Oxidative stress and the amyloid beta peptide in Alzheimer's disease. Redox Biol 14, 450-464.
[28] Christen Y (2000) Oxidative stress and Alzheimer disease. Am J Clin Nutr 71, 621S-629S.
[29] Smith D G, Cappai R, Barnham K J (2007) The redox chemistry of the Alzheimer's disease amyloid beta peptide. Biochim Biophys Acta 1768, 1976-1990.
[30] Ahmad W, Ijaz B, Shabbiri K, Ahmed F, Rehman S (2017) Oxidative toxicity in diabetes and Alzheimer's disease: mechanisms behind ROS/RNS generation. J Biomed Sci 24, 76.
[31] Ganguly U, Kaur U, Chakrabarti S S, Sharma P, Agrawal B K, Saso L, Chakrabarti S (2021) Oxidative Stress, Neuroinflammation, and NADPH Oxidase: Implications in the Pathogenesis and Treatment of Alzheimer's Disease. Oxid Med Cell Longev 2021, 7086512.
[32] Huang W J, Zhang X, Chen W W (2016) Role of oxidative stress in Alzheimer's disease. Biomed Rep 4, 519-522.
[33] Kaur U, Banerjee P, Bir A, Sinha M, Biswas A, Chakrabarti S (2015) Reactive oxygen species, redox signaling and neuroinflammation in Alzheimer's disease: the NF-kappaB connection. Curr Top Med Chem 15, 446-457.
[34] Verri M, Pastoris O, Dossena M, Aquilani R, Guerriero F, Cuzzoni G, Venturini L, Ricevuti G, Bongiorno A I (2012) Mitochondrial alterations, oxidative stress and neuroinflammation in Alzheimer&#x0027;s disease. Int J Immunopathol Pharmacol 25, 345-353.
[35] Hopperton K E, Mohammad D, Trepanier M O, Giuliano V, Bazinet R P (2018) Markers of microglia in post-mortem brain samples from patients with Alzheimer's disease: a systematic review. Mol Psychiatry 23, 177-198.
[36] Hanzel D K, Trojanowski J Q, Johnston R F, Loring J F (1999) High-throughput quantitative histological analysis of Alzheimer's disease pathology using a confocal digital microscanner. Nat Biotechnol 17, 53-57.
[37] Muramori F, Kobayashi K, Nakamura I (1998) A quantitative study of neurofibrillary tangles, senile plaques and astrocytes in the hippocampal subdivisions and entorhinal cortex in Alzheimer's disease, normal controls and non-Alzheimer neuropsychiatric diseases. Psychiatry Clin Neurosci 52, 593-599.
[38] Vehmas A K, Kawas C H, Stewart W F, Troncoso J C (2003) Immune reactive cells in senile plaques and cognitive decline in Alzheimer's disease. Neurobiol Aging 24, 321-331.
[39] Pereira J B, Janelidze S, Smith R, Mattsson-Carlgren N, Palmqvist S, Teunissen C E, Zetterberg H, Stomrud E, Ashton N J, Blennow K, Hansson 0 (2021) Plasma GFAP is an early marker of amyloid-beta but not tau pathology in Alzheimer's disease. Brain.
[40] Huat T J, Camats-Perna J, Newcombe E A, Valmas N, Kitazawa M, Medeiros R (2019) Metal Toxicity Links to Alzheimer's Disease and Neuroinflammation. J Mol Biol 431, 1843-1868.

### Example 3

Alzheimer's disease (AD) is the most common cause of dementia among elderly people, but the precise factors contributing to its etiology have not yet been determined [1]. In an aging population, dementia will present one of the greatest challenges to society in this century. The mechanisms that regulate neuronal degeneration in AD remain unclear. However, the cytopathological hallmarks of AD appear to be the deposition of extracellular amyloid-β (Abeta) plaques between neurons and the intracellular accumulation of phosphorylated (p)-Tau, which lead ultimately to profound neuronal toxicity and atrophy [2]. As Abeta plaque formation is one of the underlying mechanisms implicated in AD, prevention of such formation, particularly at early disease onset, would be an important goal to eradicate this disease.

Despite decades of research, the pathogenetic mechanisms that initiate Abeta aggregation are still largely unknown. Postmortem studies of AD human brains showed that there is a huge imbalance of oxidant-antioxidant status that leads to cell death and tissue damage [3]. Reactive oxygen species (ROS) play a pivotal role in normal cellular and signaling pathways, albeit excessive generation of ROS leads to harmful effects, including cellular and protein damage [4-6]. Oxidative stress and damage due to ROS have been implicated in the pathogenesis of AD [7]. Extensive studies have highlighted in particular the role of superoxide anion (^{∘}O₂⁻), hydroxyl radical (^{∘}OH), hydrogen peroxide (H₂O₂), and nitric oxide (NO^{∘}) in the oxidative stress-mediated neurodegeneration of AD [8,9]. The brain is more vulnerable to oxidative stress than any other organs since components of neurons, such as lipids, proteins, and nucleic acids, can be oxidized in AD. These changes in oxidative stress are associated with increased metal levels, inflammation, and Abeta peptides [10]. Postmortem analysis of human AD brains showed that iron (Fe), zinc (Zn), and copper (Cu) are associated with oxidative stress, and they accumulate in the Abeta plaques of these AD patients [11,12]. Others have also demonstrated that these metals have a very high affinity for Abeta42, a major component of amyloid plaques [13]. Notably, these three metals are cofactors in redox reactions, and they can switch between oxidation states through the Fenton mechanism to generate radicals, which catalyze the conversion of inert peptide side chains into very reactive species. For example, Cu⁺, Fe²⁺, and Zn⁺ could (i) act as cofactors with enzymes to convert inert peptide side chains to active and unstable species; (ii) react with H₂O₂ under stress conditions to generate ^{∘}OH, which in turn induces particular aggregation, ultimately leading to the formation of annular protofibrils and fibrillar Abeta aggregation [14]. This formation of structural aggregates with biophysical properties is associated with high neurotoxicity and neurodegeneration. Control or suppression of these reactions may therefore provide a valid method to slow down disease onset or Abeta accumulation and associated damage.

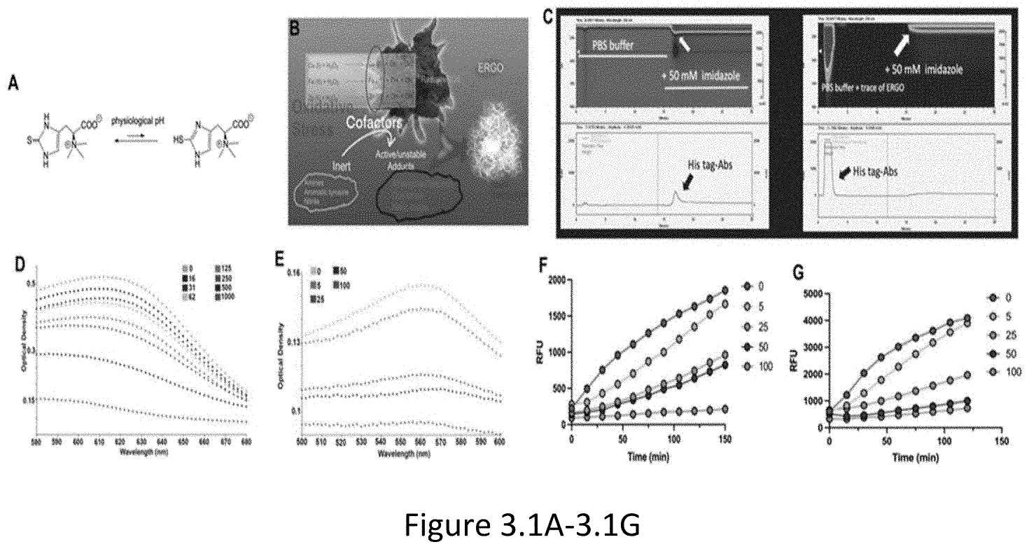

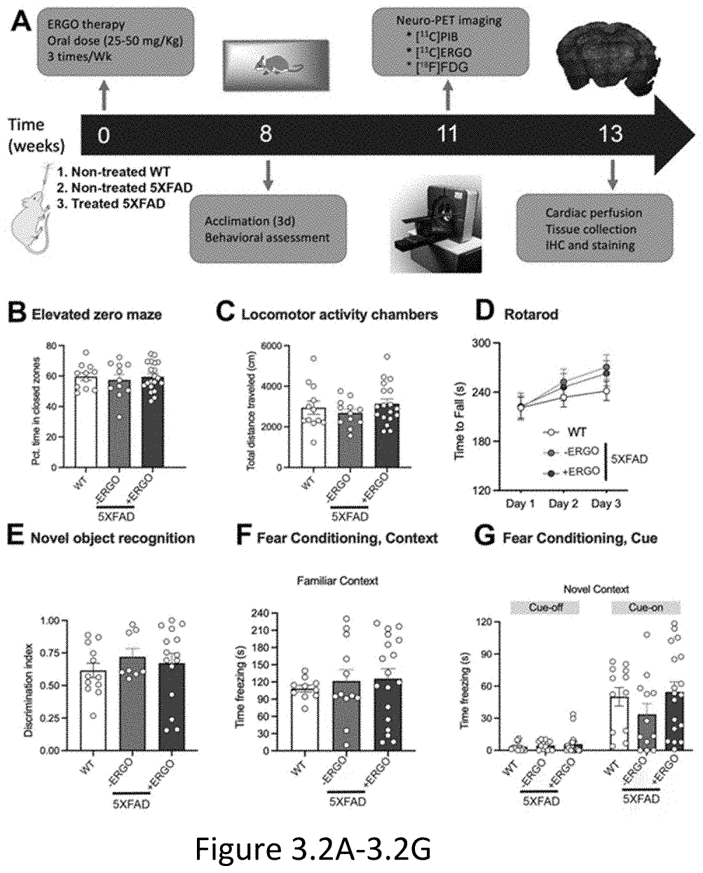

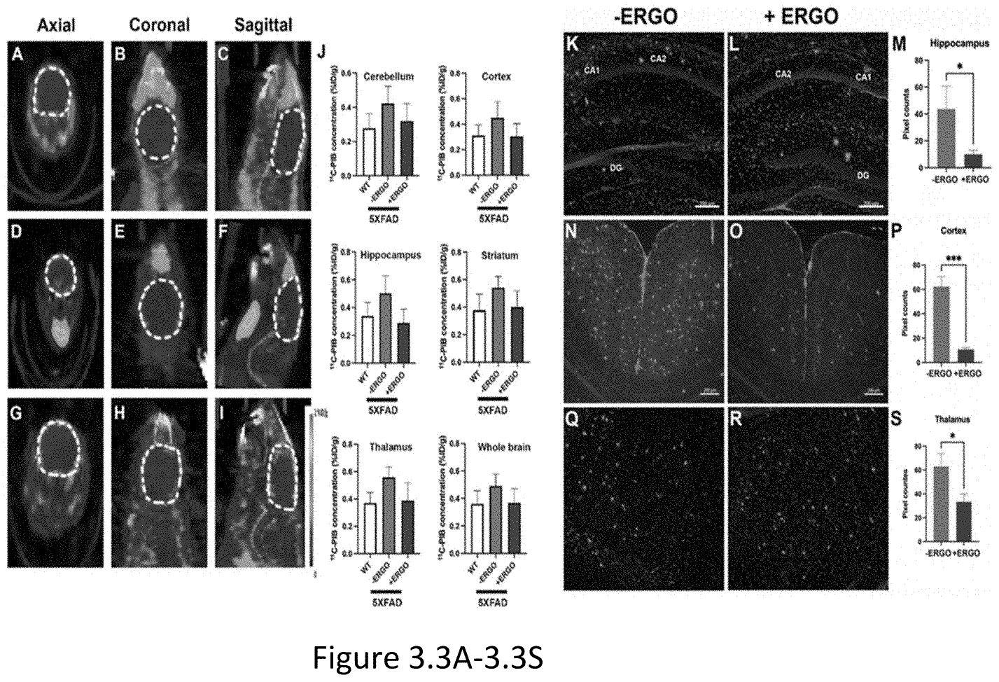

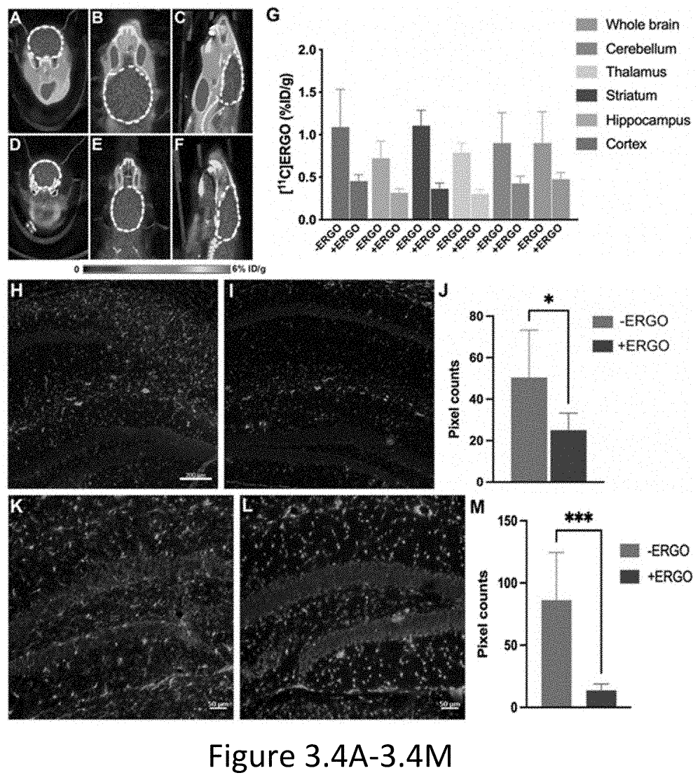

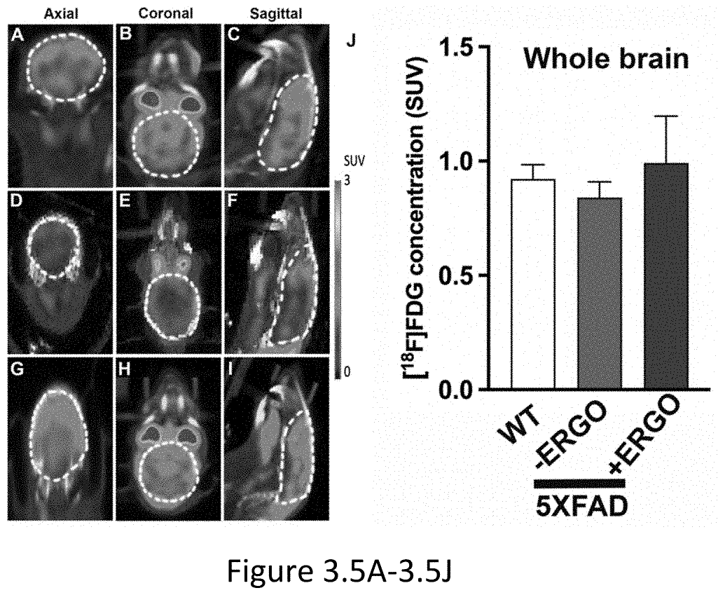

Mushrooms are a rich source of four bioactive compounds essential to human health, including selenium [15,16], vitamin D [17,18], glutathione [19], and ergothioneine (ERGO) [20,21]. Except for Vitamin D, the remaining compounds are antioxidant agents. Accumulating evidence indicates that ERGO in particular is a physiological antioxidant cytoprotectant [22-25], existing in the body as a water-soluble zwitterion (FIG. 3.1A). ERGO protection against cytotoxicity elicited by Cu²⁺, H₂O₂, Fe and sodium nitrite (NaNO₂) [26-29] is derived from its conspicuous affinity for metal cations, such as Fe and Cu, permitting capture and neutralization of associated radicals [30]. Humans obtain ERGO through the diet, but blood ERGO concentration decreases significantly with age, and markedly lower levels have been found in individuals with mild cognitive impairment compared to healthy counterparts. This observation supports the concept that ERGO deficiency acts as a risk factor for neurodegeneration [31].

To test the biodistribution and pharmacokinetics of ERGO in vivo, we recently reported the development of an [¹¹C]ERGO PET radioligand [32]. Our study suggested that this zwitterion molecule is very easy to formulate for oral administration since it is very polar. Based on our in vivo PET imaging data, we hypothesized that ERGO could serve as an ideal antioxidant for AD since it can be distributed to the brain by an oral route. ERGO uptake is very high in the small intestine, suggesting that there are abundant receptors (OCTN1 (novel organic cation transporter 1)) in the gut that would shuttle the compound to the circulation where it will be distributed to the brain mediated by OCTN1 receptors expressed in the brain parenchyma.

Since ROS and metal products have a profound impact on downstream Abeta pathology, in this work, we investigated whether consumption of ERGO benefits the brain of 5XFAD mice. Our working hypothesis is that this dual ROS/metal scavenging antioxidant will serve as a potent radical inhibitor to prevent Abeta aggregation (FIG. 3.1B). The data showed a promising effect of longitudinal intake of ERGO when testing the compound on 5XFAD mice. The treated cohort of 5XFAD mice showed modest improvement in cognition. In contrast, we observed a marked reduction of biomarkers related to AD pathogenesis at the molecular level using PET imaging. Particularly, Abeta burden was substantially reduced, and glucose metabolism is rescued in treated young 5XFAD mice. Further, this work also suggests that the [¹¹C]ERGO PET radioligand could potentially be used to track oxidative stress levels as a biomarker to assess the efficacy of AD therapy.

### Results

### ERGO is a Metal Scavenger

To demonstrate that ERGO has a high affinity for metals, we developed an assay utilizing 2D-HPLC equipped with a nickel column. In principle, nickel is a transition metal like Fe, Cu and Zn. The nickel column can immobilize hexahistidine-tag (His-tag) recombinant proteins, such as recombinant antibodies harboring a His tag (His-Abs). In our experimental design, nickel forms a substantial coordination equilibrium with histidines via the imidazole ring structures to form a stable metal/ligand complex. This assay uses two different buffer reservoirs, a wash buffer (PBS) and an elution buffer (PBS containing 50 mM imidazole). To remove the His-Abs (14 KDa) from the nickel column, the washing buffer was switched to the elution buffer and the His-Abs dislodged from the column were detected by the 2D-HPLC (FIG. 3.1C, left). In a different phase of the experiment, the His-Abs solution was spiked with ERGO (14.5 mM) prior to injection into the HPLC system. As a result, the His-Abs were eluted from the nickel column immediately by the wash buffer (FIG. 3.1C, right). Overall, the data suggest that with a strong affinity with metals, ERGO competes with the His-tag and purges the His-Abs from the column. The integrity of the eluted His-Abs was collected and confirmed by orbit trap mass spectrometry.

We also assessed ERGO's transition metal scavenging using cupric ion chelating (CIC) and ferrous ion chelating (FIC) assays. In these experiments, pyrocatechol violet or ferrozine forms a complex with free Cu(II) or Fe(II), respectively, resulting in a chromophore with a strong absorbance signal. The pyrocatechol violet-COI) complex has an absorbance lambda max (λₘₐₓ) at 630 nm, and ferrozine-Fe(II) has a λₘₐₓ at 562 nm. In the presence of ERGO, fewer free metals are available to form these complexes, resulting in less absorbance. Data in FIG. 3.1D, 3.1E show that the signals decreased as the concentrations of ERGO increased in a dose-dependent fashion.

### ERGO is a ROS Scavenger

We used a commercially available DCFH-DA (2′,7′-dichlorodihydrofluorescein diacetate) probe to assess the ROS scavenging power of ERGO in HeLa and neuroblastoma SH-SY5Y cells. Upon penetrating the cytoplasm, DCFH-DA is deacetylated by endogenous esterases to a non-fluorescent intermediate, followed by oxidation by free radicals. This generates a continuous and extended conjugation system, resulting in the production of highly fluorescent DCF (2′ 7′-dichlorofluorescein). In an aqueous condition, DCF has a maximal excitation and emission of 495 nm and 529 nm, respectively. Well-adhered HeLa or SH-SY5Y cells previously incubated with DCFH-DA were exposed to a commercial ROS initializer and ERGO concentrations ranging from 0-100 mM. The data in FIG. 3.1F, 3.G suggest that ERGO scavenges ROS products, leading to the quenching of the fluorescence signal of the DCF probe. The scavenging effect is dose-dependent; starting from 5 mM, ERGO attenuates the fluorescence significantly. At a dose of 100 mM, the fluorescence is nearly negligible. Each point in the assay was measured in pentaplicate, p<0.0001.

### Timeline of the Therapy and Processing

To address our primary question whether longitudinal consumption of ERGO as a potent antioxidant benefit the brain, three cohorts of animals (n=12 non-treated WT control, n=12 non-treated 5XFAD, and n=18 ERGO-treated 5XFAD) were used (FIG. 3.2A). Starting at the age of 8 weeks, the animals were treated with high doses of ERGO solution (25-50 mg/Kg) formulated in double distilled (dd) water via oral gavage three times a week over the course of eight weeks. To reduce bias in the behavioral experiments with regard to the potential stress during gavage treatment, non-treated animals underwent the identical gavage process with the vehicle, water, only. The efficacy of the therapy was evaluated by behavioral testing, followed by three different non-invasive PET imaging sessions to assess biomarkers related to AD pathology. In addition to assessing Abeta levels and glucose metabolism using the [¹¹C]PIB and [¹⁸F]FDG probes, respectively; we also tested for the first time whether oxidative stress could be used as an indicator of response to therapy using the [¹¹C]ERGO PET radioligand. Finally, animals were euthanized, and the brains were collected for immunohistochemical analysis.

### ERGO Treatment Prevents Early Cognitive Deficits in 5XFAD Mice

At the end of the ERGO treatment, mice were transferred to the animal neurobehavior facility to acclimate for at least three days before behavioral assessment. Cognition of WT (n=12), non-treated 5XFAD (n=12) and ERGO-treated 5XFAD (n=18) mice was assessed using a variety of different assays.

We first confirmed that ERGO treatment had no adverse effects on activity or anxiety-like behavior in the animals that could potentially impact the efficacy and translatability of ERGO as an interventional strategy or that would impact interpretation of cognitive behavioral tasks. We used the elevated zero maze (EZM) to assess anxiety-like behaviors of 5XFAD mice following ERGO treatment. All mice had equivalent exploration in the maze as assessed by total distance travelled (F_{(2, 39)}=0.82, p=0.45). Anxiety-like behavior in this task is indexed via exploration of brighter, open areas of the maze compared to darker, enclosed regions, perceived as “safe zones” [33]. Each group spent close to 60% of their time exploring the closed zones suggesting normal exploratory activity (F_{(2, 39)}=0.19, p=0.82) (FIG. 3.2B). Exploratory activity in a novel environment was further assessed in locomotor activity chambers across a 30-min. session. Although 5XFAD mice are sometimes reported to be hyperactive at 8 months and older, we observed similar exploration in all three groups (F_{(2, 39)}=0.80, p=0.46) (FIG. 3.2C). All mice were able to learn the rotarod as evidenced by increasing latencies to fall across the three days of testing (F_{2,63}=24.13, p<0.0001) suggesting intact cerebellum-dependent motor learning. There were no differences among the groups or day x group interaction (Fs<1.20, p>0.32) (FIG. 3.2D). Exploration in the empty novel object arena was equivalent among groups (F_{2, 37}=0.24, p=0.78, data unavailable for 2 mice due to computer tracking error). Mice that explored objects for less than 10 seconds in total or explored any single object for less than 5 seconds were not included in recall analyses (3 non-treated 5XFAD, 2 treated 5XFAD). For all other mice, initial exploration did not vary according to location (left/right) or group (Fs<1.80, p>0.18) and ranged from 20.1-77.8 seconds in WT, 14.7-124.9 seconds in non-treated 5XFAD and 11.5-124.6 seconds in ERGO-treated 5XFAD. During the final test trial data from one additional 5XFAD mouse was excluded because it spent 0 seconds within the target zone. However, most mice spent more time in proximity to the novel object than the familiar object, but the discrimination index did not vary among the groups (F_{2, 32}=0.48, p=0.62) (FIG. 3.2E).

Mice were then trained to associate a series of three small (0.5 mV, 1 second) electric shocks with the termination of a 30-second auditory cue in the conditioned fear task. Recall of the shocks was tested by indexing time spent freezing (a fear response) when mice were exposed to the same testing context, or to the previously-paired tone within a novel testing context, 24 and 25 hours following training, respectively. All mice showed a strong memory of the testing environment as evidenced by similar levels of freezing when returned to the original testing chambers (Kruskal-Wallis statistic=0.54, p=0.77) (FIG. 3.2F). When mice were exposed to a novel context (plastic lined walls and floor and vanilla odor) exploratory activity resumed, indicating that prior freezing, and therefore learning was specific to the testing context. In contrast, when the cue was re-introduced, WT control and ERGO-treated 5XFAD mice showed improved recall compared to non-treated 5XFAD, as evidenced by increased freezing during presentation of the cue (FIG. 3.2G). Although all mice increased freezing time when the cue was played compared to the “tone-off” portion of the trial (F_{(1,37)}=65.95, p<0.0001), performance was not equivalent between the groups. ERGO-treated 5XFAD mice strongly resembled performance of control WT mice with freezing close to 50% of the time that the cue was played (p<0.0001), compared to non-treated 5XFAD control animals that froze only approximately 25% of the time (p<0.05).

### Longitudinal Consumption of ERGO Mitigates Abeta Aggregation in Young 5XFAD Mice

Non-invasive, in vivo, and robust assessment of Abeta levels in the brains of treated and non-treated 5XFAD mice were performed using the [¹¹C]PIB PET probe, the chemical structure of which has been described elsewhere [34]. All animals were injected via the tail vein with the same radioisotope dose (˜15 MBq of [¹¹C]PIB) and volume (100 μL/mouse). To test the preventive effect of ERGO, we treated 5XFAD mice starting at the age of 2-month-old. The idea of this work is based on the premise that young 5XFAD mice do not develop Abeta deposits/plaques in the brains until 2-month-old [35]. We found a steady increase of PET signal in non-treated 5XFAD mice (n=5), at the age of 4-month-old (FIG. 3.3D, 3.3E, 3.3F), suggestive of the presence of extracellular Abeta plaques. The PET signals obtained from ERGO treated, age-matched 5XFAD (n=7) (FIG. 3.3G, 3.3H, 3.3I) were very weak, and resembled the background signal found in WT mice (n=4) (FIG. 3.3A, 3.3B, 3.3C). We co-registered the PET/CT image to an MRI template for quantitative PET analysis of regional uptake in the brain. The volumetric region-of-interest was drawn around the cortex, hippocampus, striatum, thalamus and cerebellum, in addition to the whole brain. The concentration of the injected [¹¹C]PIB in PET imaging expressed as percentage of injected dose per gram tissue (% ID/g), is significantly higher in every subregion of the brain (p<0.05) in non-treated 5XFAD compared to treated counterparts (˜30-50%) (FIG. 3.3J).

To confirm the in vivo observations, animals were perfused, and the brain sections were stained with anti-Abeta antibody, 6E10. Our staining data showed that in non-treated 5XFAD mice, Abeta was highly expressed in the brain regions typically susceptible to AD, including the hippocampus, cortex, and the thalamus (FIG. 3.3K, 3.3N, 3.3Q) at approximately 4 months of age. In particular, we observed a clear difference in the hippocampus, pyramidal cortex and thalamus of non-treated compared to ERGO-treated 5XFAD mice (FIG. 3L, 3.30, 3.3R). The pixel counts were further quantified between cohorts with ImageJ; this demonstrated a 4-fold (p<0.05), 5-fold (p=0.0004) and 2-fold (p<0.05) decrease in Abeta level in the regions of hippocampus, pyramidal cortex and thalamus, respectively, in treated 5XFAD mice (FIG. 3.3M, 3.3P, 3.3S).

[¹¹C]ERGO PET radioligand detects oxidative stress reduction in ERGO-treated 5XFAD mice Recently, our [¹¹C]ERGO PET radioligand has been demonstrated as an imaging biomarker for oxidative stress in a mouse model of lipopolysaccharides (LPS) [32] and 5XFAD mice [36]. Thus, in this study, [¹¹C]ERGO PET radioligand was used to assess the fluctuation in oxidative stress with respect to ERGO therapy. We observed very modest reduction in PET signal in a 20-min dynamic scan after intravenous (IV) injection, pertaining to oxidative stress in the brains of treated mice (FIG. 3.4D, 3.4E, 3.4F) compared to non-treated 5XFAD mice (FIG. 3.4A, 3.4B, 3.4C). This observation could be due to the fact that the animals were treated with high doses of ERGO during gavaging. Although we stopped treating mice with ERGO weeks before injection of the [¹¹C]ERGO PET radioligand, the residual ERGO from the last treatment could have blocked the majority of the binding sites and thus resulted in less-to-none PET signal. Nevertheless, post-imaging data analysis revealed that [¹¹C]ERGO uptake differentiated the level of oxidative stress of non-treated 5XFAD mice from age-matched treated 5XFAD mice (FIG. 3.4G) (p<0.05).

We also found that other key inflammation-related protein markers were positively correlated with [¹¹C]ERGO PET imaging data, supporting [¹¹C]ERGO PET radioligand as a robust marker for neuroinflammation in AD. In this study, after the PET imaging, animals (non-treated 5XFAD (n=3) and ERGO-treated 5XFAD mice (n=5)) were perfused, and the brain sections were stained with antibodies against GFAP and IBA1 for neuroinflammatory markers astrocytes and microglia, respectively. Data in FIG. 3.4H, 3.4I, 3.4J showed that the level of GFAP-positive astrocytes in the hippocampus was reduced by nearly 50% in the treated group (FIG. 3.41) compared to non-treated 5XFAD mice (FIG. 3.4H). Similarly, treating mice with ERGO resulted in nearly an 80% reduction of IBA1-positive microglia (FIG. 3.4K (non-treated 5XFAD), 3.4 L (treated 5XFAD), 3.4M), p=0.0007.

### ERGO Treatment Rescues Glucose Metabolism in 5XFAD Mice

The brain consumes a significant amount of glucose, approximately 25% of the body's glucose [37]. It is well known that glucose metabolism is diminished in the early onset of AD. Therefore, [¹⁸F]FDG PET imaging to assess glucose levels could be used for AD diagnosis. For this purpose, three cohorts of age-matched mice (6-month-old, n=3 for each cohort), including WT, non-treated 5XFAD and ERGO-treated 5XFAD (6 doses, each 50 mg/Kg during the course of 2 weeks) were studied. Afterward, animals were injected with [¹⁸F]FDG probe (15 MBq, 100 μL/mouse) and underwent 20-min dynamic PET scans and CT scans. The data in FIG. 3.5 showed that non-treated 5XFAD mice (3.5D, 3.5E, 3.5F, 3.5J) had lower glucose metabolism compared to WT mice (3.5A, 3.5B, 3.5C, 3.5J). In stark contrast, glucose levels were significantly elevated (p<0.05) in the brains of ERGO treated compared to non-treated 5XFAD mice (FIG. 3.5G, 3.5H, 3.5I, 3.5J), suggesting the rescuing role of ERGO as an antioxidant in glucose metabolism in AD.

### Discussion

There is convincing evidence demonstrating that free radical damage and oxidative stress play a pivotal role in the early onset of AD [38]. Postmortem brain tissues from AD patients have significant extent of oxidative damage associated with extracellular Abeta plaques and intracellular neurofibrillary tangles [39]. Further, metals have been described to be involved in different pathophysiological mechanisms associated with neurodegenerative diseases, including AD [40]. With the availability of magnetic resonance imaging, iron has been mapped in the brain regions usually associated with Abeta deposits in AD [41,42]. Iron accumulation in the Abeta plaques has also been detected by MRI [43]. Another factor that makes the brain more susceptible to oxidation in AD is the presence of transition metals [44], such as Fe, Zn and Cu [45,46]. These metals serve as a catalyst for the redox-generated free radicals, such as hydroxyl radicals, which have been proposed to initiate Abeta aggregation [47]. Free metal ions have been detected with abnormally high concentration in the ageing brain, as well as during several neurodegenerative disorders [11,48-50]. In AD brains, endogenous metals like Fe, Zn, Cu and Fe are found at a higher concentration in Abeta plaques compared to healthy brains [51]. For example, high levels of Cu (400 μM) and Zn (1 mM) were reported in AD brain compared to 70 μM and 350 μM, respectively in healthy brain [11,52].

Taking all these factors into account, it is apparent that reducing levels of ROS levels and/or neutralizing transitions metals which are catalysts for ROS production, would benefit the brain. ROS- and transition metal-chelating therapy [53,54] have been tested in clinical trials for the treatment of AD using antioxidants and chelators, respectively. These trials show that dietary uptake of antioxidant supplements, like vitamin E or both vitamins C and E may lower the risk of AD [55,56]. Likewise, clinical trials using clioquinone derivatives as metal chelators showed reduced cerebrospinal fluid (CSF) Abeta-42 concentration [57]. Despite some initial success, there have been several issues associated with such antioxidants and chelators which have become the subject of debate, including toxicity and the overall limited ability to cross the blood-brain barrier (BBB) [58]. In that regard, ERGO has more advantage given it is a dual ROS/metal scavengers, which can be distributed to the brain independent of the BBB. In this project, we show that treating 5XFAD mice with high doses of ERGO resulted in reduced Abeta burden, oxidative stress and rescue glucose metabolism, and with some modest improved cognition.

This study shows that imaging biomarkers associated with AD pathogenesis may offer a distinct opportunity for monitoring the response to therapy. Interestingly, we observed both cognitive and molecular benefits of ERGO treatment in 4-month-old 5XFAD mice prior to the onset of significant behavioral deficits, and at least 2 weeks after ERGO treatment had been stopped. This suggests either a lasting benefit of the compound, or at least a delay in onset of genotype-associated behavioral and molecular changes. Importantly, ERGO did not have any detrimental impact on activity levels, anxiety-like behaviors or motor learning which would limit its utility as a therapeutic intervention. All three groups of mice were able to learn the cue-context-shock associations as evidenced by increased freezing behaviors during the two test trials. However, learning was weaker in the non-treated 5XFAD mice which appeared to freeze at approximately half of the rate of WT and ERGO-treated 5XFAD animals during the presentation of the cue (tone) in the novel context. This task requires functional connections between hippocampus, frontal cortex, cingulate cortex as well as the amygdala, which may be particularly important in cue retrieval which was preserved by ERGO treatment [59]. Context retrieval is thought to be more directly dependent on hippocampal inputs. While we did not demonstrate a role for ERGO treatment in context retrieval, it should be noted that at this age there were also no deficits in the non-treated 5XFAD mice on this portion of the task. Similarly, we detected no clear deficits in the 5XFAD mice on the novel object recognition task which is also thought to be highly dependent on intact hippocampal function. However, we used a protocol in which the exposure and test trials were separated by approximately 2 minutes. It is possible that had we imposed a longer delay that recall would have been impaired in the 5XFAD mice which would then have allowed a clearer evaluation of any potential beneficial effects of ERGO treatment. It will therefore be important to further assess potential impacts of short- or longer-term ERGO treatment in more cognitively demanding tasks, or in older animals in which cognitive deficits are more widespread.

### Materials and Methods

### ERGO Formulation

L-ERGO was purchased from Cayman Chemical (Ann Arbor, Mich.) and used without any further purification, albeit the material was authenticated and confirmed via mass spectrometry and spectrophotometry before use. The compound was diluted in dd. water at room temperature at a concentration of 50 mg/Kg as a fresh stock solution for every gavage treatment.

### Mass Spectrometry Analysis

Liquid Chromatography Mass Spectrometry (LC-MS) was performed on the sample using a Waters Aquity HPLC and a Thermo Fisher LTQ-Orbitrap XL. The MS was operated in FT mode in positive ion mode with 60 k resolution scanning between 200 and 2000. About 10 μL of the sample were injected onto a symmetry 300 C18 3.5 μm, 2.1×100 mm column, and mobile phases A) 0.2% formic acid and 0.05% TFA in water/acetonitrile (9/1) and B) 0.2% and 0.05% TFA in acetonitrile/water/isopropanol (4/1/5) with a flow rate of 300 μL/min. The gradient held steady at 100% phase A for 1 min before changing to 100% phase B at 10 min and holding for 1 min before returning to the starting conditions and re-equilibrating for 3 min. The data collected was examined using Qual Browser software.

### ROS Measurement

On day 1, HeLa cells or SH-SY5Y cells were seeded into a 96-well plate with a density of 10,000 cells/mL. On day 2, the DCFH-DA probe (Cell Biolabs, Inc., San Diego, USA) was formulated in cell culture media (Gibco Dulbecco's Modified Eagle Medium (DMEM) and dispensed to allocated wells at a final concentration of 1×, as recommended by the manufacturer. The plate was then incubated in the dark for 1 h at 37° C. before excess probe was removed, and the cells were washed repeatedly 3 times with Dulbecco's Phosphate-Buffered Saline (DPBS). Then, ERGO solution was added to the designated wells at concentrations ranging from 5 to 100 mM. The free radical initiator solution was prepared at a concentration of 1× as per manufacturer's recommendation and added to all wells. Immediately afterwards, fluorescence measurement began using a microplate reader (Biotek Industries, Agilent Technologies, Winooski, Vt., USA) with an excitation and emission wavelength of 485 nm and 528 nm, respectively. Each ERGO concentration was measured in pentaplicate.

### Metal Scavenging Assays

### Nickel Assay

ERGO's metal-binding affinity was tested via a 2D-HPLC equipped with a nickel-based HisTrap HP column (Cytiva, Marlborough, Mass., USA). The 2D-HPLC was performed with a Hitachi LaChrom Elite L-2455 Diode Array Detector linked to a Hitachi L-2130 pump (Hitachi High-Technologies, Corp., Tokyo, Japan). Two reservoirs of the mobile phase, including PBS and PBS with 50 mM imidazole were programmed so that the first 15 min of the run will be covered with 100% PBS, followed by switching to 100% of the eluting buffer which is comprised of PBS with 50 mM imidazole. In a typical experiment, histidine-containing camelid antibodies (250 ng, MW 14 KDa) dissolved in 200 μL PBS were first injected into the HPLC system. In this setup, the histidine-containing camelid antibodies were immobilized in the nickel column exclusively during the first 15 min with PBS buffer. However, when the mobile phase was changed to PBS with large concentration of imidazole, which will compete and displace His-tag from camelid antibodies resulted in the antibody elution. In another experiment, the antibody was injected as described above but with the addition of ERGO (50 nM). Right at the beginning of the run, His-tag camelid antibodies were eluted, indicating that ERGO competes with the His tag for nickel resulting in an early washout of the antibodies. All analyses were performed via the EZChrom Elite software package (Agilent Technologies, Winooski, Vt., USA).

### Ferrous Ion Chelating (FIC) Assay

The iron chelating activity of ERGO was determined using a FIC assay, which was obtained from Zen-Bio Inc. (Durham, N.C.). The assay was done on a 96-well plate with triplicated samples, standards, background, and max absorbance wells. All of the assay absorbance values were subtracted from the average background absorbance, comprising of water and assay buffer (1:1). Maximal absorbance was measured by averaging the absorbance values of 3 wells containing only FeSO4 and assay buffer. For the testing wells, each contained FeSO4 (50 μL) and ERGO samples at different concentrations, including 0, 5, 25, 50, and 100 mM. Control wells contained EDTA standards and a solution of FeSO4 (50 μL), except for the background wells. The assays were started by adding ferrozine solution (100 μL, 1×) to each well using a multichannel pipet, with the final volume of each well being 200 μL. The assaying plate was incubated at room temperature for 10 minutes then the plate was read at 562 nm using a Biotek Synergy 4 plate reader. Absorbances were averaged, and the background absorbance was subtracted from the sample and maximum averages (Absₜₑₛₜ and Absₘₐₓ). The ferrous iron chelating percentage was then calculated using the following equation: Ferrous ion chelating (%)=100×(Absₘₐₓ−Absₜₑₛₜ)/Absₘₐₓ.

### Cupric Ion Chelating (CIC) Assay

The copper chelating activity of ERGO was determined using a CIC assay (Zen-Bio Inc., Durham, N.C.). Assays contained triplicated samples, standards, background, and max absorbance wells. All the assay absorbance values were subtracted from the average background absorbance, comprising of water and assay buffer (1:1). Maximal absorbance was measured by averaging the absorbance values of 3 wells containing only CuSO₄ and assay buffer. For the testing wells, each contained CuSO₄ (30 μL) and ERGO samples at different concentrations, including 0, 16, 31, 62, 125, 250, 500, and 1000 mM. Control wells contained EDTA standards and a solution of CuSO₄ (30 μL), except for the background wells. The plate was incubated at room temperature for 5 minutes. Assays were started by adding pyrocatechol violet solution (8.5 μL, 1×) to each well, with the final volume of each well being 268.5 μL. The assaying plate was incubated at room temperature on a shaker for 10 minutes, followed by an additional 10-minute incubation at room temperature without shaking. The plate was then read at 632 nm using a Biotek Synergy 4 plate reader. Absorbances were averaged, and the background absorbance was subtracted from the sample and maximum averages (Absₜₑₛₜ and Absₘₐₓ). Cupric ion chelating percentage was then calculated using the following equation: Cupric ion chelating (%)=100×(Absₘₐₓ−Absₜₑₛₜ)/Absₘₐₓ.

### Animals

A colony of 5XFAD mice obtained from Jackson Laboratories was maintained by crossing with WT C57BL/6J as we reported in the past [60]. The animals were genotyped by polymerase chain reaction (PCR) using DNA obtained from tail or ear tissue samples. After PCR amplification, DNA product was analyzed using a 1% agarose gel; amyloid precursor protein (APP) transgene=377 bp, and presenilin 1 (PSEN1) transgene=608 bp. 5XFAD mice were maintained as heterozygous. Animals were treated with ERGO formula beginning at the age of 2-month-old. Animal experiments were conducted in accordance with the guidelines established by the Vanderbilt University's Institutional Animal Care and Use Committee (IACUC) and the Division of Animal Care and approved by Vanderbilt IACUC.

### Gavage Treatment

Mice (2-month-old) were dosed (50 mg/Kg, less than 100 μL) by means of oral gavage. The procedure involves passing a reusable oral gavage needle through the mouth and placing it atop of the esophagus of an awake animal in the way to encourage the animal to swallow the formulation voluntarily. The curvature of the syringe along with the extra smooth round ball stainless steel tip ensured minimal discomfort to the treated animals. Since the gavage procedure involved restraining of the animal, it might cause stress [61], which is a potential confounding experimental endpoint in behavioral assessments. To ameliorate the potential discrepancy between treated and non-treated cohorts, the later underwent the same oral gavage process, but were given only the vehicle, sterilized water (100 μL).

### Behavioral Experiments

The primary objective of this work was to answer whether ERGO treatment benefits the brain and improves cognition. At the end of ERGO-based therapy, animals were transferred to the Vanderbilt Neurobehavioral Laboratory to acclimate for at least 3 days before testing.

### Elevated Zero Maze (EZM)

We recorded exploratory activity in open versus closed zones of a standard EZM during a 5-min trial. At the start of the trial, mice were placed in an open zone of the maze and allowed to explore freely while being videotaped from above. The floor of the maze is 5 cm wide. Closed zones had walls of approximately 30 cm height and light levels of 215-280 lux depending on where they were measured. Open zones had a small lip of ˜0.5 cm and light levels of 349-469 lux. Trials were observed by an experimenter in an adjacent room.

All behavioral apparatus were cleaned with 10% ethanol solution between trials to sanitize the equipment and minimize odor trails lefts by previous animals. The animal's location within the maze and distance traveled were analyzed using AnyMaze (Stoelting Co.).

### Locomotor Activity

Exploratory locomotor activity was measured in specially designed chambers measuring 27×27 cm (Med Associates), housed in sound-attenuating cases over a 30-min period. Horizontal and vertical activity within the chambers were automatically recorded via the breaking of infrared beams.

### Rotarod

Neuromuscular ability and motor learning were assessed using a standard rotarod (Ugo Basile). Mice were placed on a 6-cm wide section of a ridged rod that rotated slowly. The rod began rotating at 4 rpm and ramped up to a maximum speed of 40 rpm by 4 min (total test time 5 min, max speed for final minute). Mice were allowed to complete 3 trials per day on 3 consecutive days. Time to fall was recorded automatically when mice fell from the rod onto a base plate.

### Novel Object Recognition

Mice were first allowed to habituate to an open arena for 5 min (white acrylic box, approximately 40 cm²) located under a camera to record position and movement during trials. Immediately following this acclimatization phase, each mouse was removed from the arena which was cleaned with 10% ethanol, and two identical objects were placed in the arena in the center of each arena half. Mice were returned to the arena and allowed to freely explore the objects for 6 min. Mice were then once again removed from the arena, which was again cleaned with 10% ethanol. A third identical exemplar of the familiar object was then added to the arena with one novel object. Position of the novel object was balanced across groups. Exploration of the two objects was permitted for 6 min until the trial was terminated. Preference for either object was inferred from exploratory proximity which was recorded automatically using AnyMaze using a target area comprising a circle with diameter 2-cm larger than the target objects. A recognition index was calculated to assess preference for the novel versus the familiar object as time in proximity to the novel object (T_{N}) divided by time spent in proximity to either novel or familiar (T_{F}) objects (T_{N}/(T_{N}+T_{F})).

### Fear Conditioning

Mice were placed in a sound-attenuating chamber with a wire grid floor capable of transmitting an electric shock. All movements were monitored by cameras fixed to the inside of the doors. Training trials were 8 min during which time a 30-second tone was played three times, that co-terminated with a small shock delivered through the metal floor (1 s, 0.5 mA). Following training mice were transferred to a holding cage in a second ante-chamber and were not reintroduced to (naïve) cage-mates until all the mice had undergone training trials. Twenty-four hours following the training trial the mice were exposed to the same chamber to assess memory for the testing context (4-min trial, no tone, no shock). Mice were tested in the same chambers, in the same test order and under the same lighting and other experimental conditions as training. One hour following the context retrieval trial, the mice were tested again in a novel context for which a second identical chamber was used but it was altered by using a white plexiglass wall and floor insert and a 10% vanilla scent placed in a weigh dish within the outer chamber (not accessible to the mouse). During this 4-min trial the previously exposed tone was played during the final 2 min but there was no shock administered. Freezing behavior was monitored automatically.

### Dynamic PET Imaging

The dynamic acquisition was divided into twelve 5-second frames, four 60-second frames, five 120-second frames, three 5-min frames, and six 10-min scans. The data from all possible lines of response (LOR) were saved in the list mode raw data format. The raw data were then binned into 3D sinograms with a span of 3 and ring difference of 47. The images were reconstructed into transaxial slices (128×128×159) with voxel sizes of 0.0815×0.0815×0.0796 cm³, using the MAP algorithm with 16 subsets, 4 iterations, and a beta of 0.0468. For anatomical co-registration, immediately following the PET scans, the mice received a CT scan in a NanoSPECT/CT (Mediso, Wash. D.C.) at an X-ray beam intensity of 90 mAs and x-ray peak voltage of 45 kVp. The CT images were reconstructed into 170×170×186 voxels at a voxel size of 0.4×0.4×0.4 mm³. The PET/CT images were uploaded into Amide software (www.sourceforge.com), co-registered to an MRI template made in-house, and volumetric regions-of-interest were drawn around the cortex, hippocampus, striatum, thalamus, and cerebellum in addition to the whole brain. The PET images were normalized to the injected dose, and the time-activity-curves (TACs) of the mean activity within the ROIs were estimated for the entire duration of the scans.

### Immunohistochemistry (IHC)

Brains embedded in OCT were cut into sagittal sections (10 μm) using a Tissue-Tek cryostat and mounted onto charged glass slides. Prior to staining, slides were washed with PBS (10 min); then, they were treated with blocking buffer (5% normal goat serum, 0.2% Triton X-100, 0.5% bovine albumin in PBS) for 1 h at room temperature. The treated sections were then incubated overnight at 4° C. with primary anti-GFAP antibody (1:100 dilution, Biolegend San Diego, Calif., USA). Slides were washed with PBS (3×) for 10 min each, the sections were subsequently incubated with secondary antibody goat anti-mouse Alexa Fluor 488 (1:200 dilution, Thermo Fisher Scientific, Carlsbad, Calif., USA) for 30 min at room temperature. The sections were then washed with PBS twice for 10 min and once for 30 min, and coverslipped with an antifade mounting medium (Vector Laboratories, Burlingame, Calif.) before observation under a fluorescence microscope.

### Data Analysis.

Quantitative analysis of the PET imaging and IHC data was performed using imageJ software. The data was imported to GraphPad Prism version 9 for Mac (GraphPad Software, San Diego, Calif., USA) for statistical analysis. Differences between groups were tested using unpaired t-test.

Behavioral data were analyzed using Prism 9 for Mac OS. Single outcome measures for elevated zero maze (EZM) and locomotor activity chambers were analyzed using Univariate ANOVA with Tukey's multiple comparisons post hoc tests following significant omnibus ANOVA. Fear conditioning data were analyzed using non-parametric approaches because data were not normally distributed (Brown-Forsythe test p<0.05). We therefore used Kruskal-Wallis test for single dependent variable (freezing in familiar context). Data were first assessed for significant effects of sex on all outcomes. Since there were no differences observed, data for male and female animals were combined.

Data are given as mean±standard error of the mean (SEM). Different levels of significance were described as *p<0.05, **p<0.01, and ***p<0.0001.

### Conclusion

The data obtained from this work demonstrated the potential use of ERGO for treating AD. It is worth noting that the concentration of ERGO used in this therapy and the assay for scavenging ROS is relatively high. Since ERGO is a natural product, it does not have therapeutic efficacy on the same par as pharmaceutical drugs. Two approaches we envision for future applications. One of those would be focusing on prevention. With a high concentration of ERGO in dietary sources, such as in mushrooms, it is anticipated that consumption of mushroom, in the long term, benefits the brain, and it could be helpful for the prevention of AD. Another approach perhaps focuses on converting this natural antioxidant into therapeutic drugs. A general structure-activity relationship (SAR) study conducted by modifying the chemical genetics surrounding ERGO could lead to a potent agent with improved efficacy.

### REFERENCES FOR EXAMPLE 3

1. Ramos-Rodriguez, J. J.; Pacheco-Herrero, M.; Thyssen, D.; Murillo-Carretero, M. I.; Berrocoso, E.; Spires-Jones, T. L.; Bacskai, B. J.; Garcia-Alloza, M. Rapid beta-amyloid deposition and cognitive impairment after cholinergic denervation in APP/PS1 mice. J Neuropathol Exp Neurol 2013, 72, 272-285, doi:10.1097/NEN.0b013e318288a8dd.
2. Selkoe, D. J. Alzheimer's disease. Cold Spring Harb Perspect 81012011, 3, 1-16.
3. Gella, A.; Durany, N. Oxidative stress in Alzheimer disease. Cell Adh Migr 2009, 3, 88-93, doi:10.4161/cam.3.1.7402.
4. Knock, G. A.; Ward, J. P. Redox regulation of protein kinases as a modulator of vascular function. Antioxid Redox Signal 2011, 15, 1531-1547, doi:10.1089/ars.2010.3614.
5. Son, Y.; Kim, S.; Chung, H. T.; Pae, H. O. Reactive oxygen species in the activation of MAP kinases. Methods in Enzymology 2013, 528.
6. Verbon, E. H.; Post, J. A.; Boonstra, J. The influence of reactive oxygen species on cell cycle progression in mammalian cells. Gene 2012, 511, 1-6, doi:10.1016/j.gene.2012.08.038.
7. Chen, X.; Guo, C.; Kong, J. Oxidative stress in neurodegenerative diseases. Neural Regeneration Research 2012, 7, 376-385.
8. Van Dyke, K. The possible role of peroxynitrite in Alzheimer's disease: a simple hypothesis that could be tested more throughly. Medical Hypotheses 1997, 48, 375-380.
9. Xie, Z.; Wei, M.; Morgan, T. E. Peroxynitrite mediates neurotoxicity of amyloid beta-peptide-42- and lipopolysaccharide-activated microglia. J Neurosci 2002, 22, 3484-3492.
10. Chen, Z.; Zhong, C. Oxidative stress in Alzheimer's disease. Neurosci Bull 2014, 30, 271-281.
11. Lovell, M. A.; Robertson, J. D.; Teesdale, W. J.; Campbell, J. L.; Markesbery, W. R. Copper, iron and zinc in Alzheimer's disease senile plaques. J Neurol Sci 1998, 158, 47-52, doi:10.1016/s0022-510x(98)00092-6.
12. Smith, M. A.; Harris, P. L.; Sayre, L. M.; Perry, G. Iron accumulation in Alzheimer disease is a source of redox-generated free radicals. Proc Natl Acad Sci USA 1997, 94, 9866-9868, doi:10.1073/pnas.94.18.9866.
13. Pohanka, M. Copper and copper nanoparticles toxicity and their impact on basic functions on the body. Bratsl Med 2019, 120, 397-409.
14. Bolognin, S.; Messori, L.; Drago, D.; Gabbiani, C.; Cendron, L.; Zatta, P. Aluminum, copper, iron and zinc differentially alter amyloid-Abeta (1-42) aggregation and toxicity. Int J Biochem Cell Biol 2011, 43, 877-885, doi:10.1016/j.bioce1.2011.02.009.
15. Beelman, R. B.; Royse, D. J. Selenium enrichment of Pleurotus cornucopiae rolland and Grifola frondosa gray mushrooms. Int J Med Mushrooms 2006, 8, 77-84.
16. Werner, A. R.; Beelman, R. B. Growing high-selenium edible and medicinal buttom mushrooms as ingredients for functional food or dietary supplements. Int J Med Mushrooms 2002, 4, 167-171.
17. Kalaras, M. D.; Beelman, R. B.; Elias, R. J. Effects of postharvest pulsed UV light treatment of white button mushrooms on vitamin D2 content and quality attributes. J Agric Food Chem 2012, 60, 220-225.
18. Kalaras, M. D.; Beelman, R. B.; Holick, M. F.; Elias, R. J. Generation of potentially bioactive ergosterol-derived products following pulsed ultraviolet light exposure of mushrooms (Agaricus bisporus). Food Chem 2012, 135, 396-401, doi:10.1016/j.foodchem.2012.04.132.
19. Kalaras, M. D.; Richie, J. P.; Calcagnotto, A.; Beelman, R. B. Mushrooms: A rich source of the antioxidants ergothioneine and glutathione. Food Chem 2017, 233, 429-433, doi:10.1016/j.foodchem.2017.04.109.
20. Dubost, N.; Ou, B.; Beelman, R. B. Quantification of polyphenols and ergothioneine in cultivated mushrooms and correlation to total antioxidant capacity. Food Chem 2007, 105, 727-735.
21. Dubost, N. J.; Beelman, R. B.; Royse, D. J. Influence of selected cultural factors and postharvest storage on ergothioneine content of common button mushroom Agaricus bisporus. Int J Med Mushrooms 2007, 9, 163-176.
22. Borodina, I.; Kenny, L. C.; McCarthy, C. M.; Paramasivan, K.; Pretorius, E.; Roberts, T. J.; van der Hoek, S. A.; Kell, D. B. The biology of ergothioneine, an antioxidant nutraceutical. Nutr Res Rev 2020, 33, 190-217, doi:10.1017/S0954422419000301.
23. Halliwell, B.; Cheah, I. K.; Drum, C. L. Ergothioneine, an adaptive antioxidant for the protection of injured tissues? A hypothesis. Biochem Biophys Res Commun 2016, 470, 245-250, doi:10.1016/j.bbrc.2015.12.124.
24. Koh, S. S.; Ooi, S. C.; Lui, N. M.; Qiong, C.; Ho, L. T.; Cheah, I. K.; Halliwell, B.; Herr, D. R.; Ong, W. Y. Effect of Ergothioneine on 7-Ketocholesterol-Induced Endothelial Injury. Neuromolecular Med 2021, 23, 184-198, doi:10.1007/512017-020-08620-4.
25. Servillo, L.; D'Onofrio, N.; Balestrieri, M. L. Ergothioneine Antioxidant Function: From Chemistry to Cardiovascular Therapeutic Potential. J Cardiovasc Pharmacol 2017, 69, 183-191, doi:10.1097/FJC.0000000000000464.
26. Aruoma, O. I.; Spencer, J. P.; Mahmood, N. Protection against oxidative damage and cell death by the natural antioxidant ergothioneine. Food Chem Toxicol 1999, 37, 1043-1053, doi:10.1016/s0278-6915(99)00098-8.
27. Colognato, R.; Laurenza, I.; Fontana, I.; Coppede, F.; Siciliano, G.; Coecke, S.; Aruoma, O. I.; Benzi, L.; Migliore, L. Modulation of hydrogen peroxide-induced DNA damage, MAPKs activation and cell death in PC12 by ergothioneine. Clin Nutr 2006, 25, 135-145, doi:10.1016/j.clnu.2005.10.005.
28. Deiana, M.; Rosa, A.; Casu, V.; Piga, R.; Assunta Dessi, M.; Aruoma, O. I. L-ergothioneine modulates oxidative damage in the kidney and liver of rats in vivo: studies upon the profile of polyunsaturated fatty acids. Clin Nutr 2004, 23, 183-193, doi:10.1016/50261-5614(03)00108-0.
29. Markova, N. G.; Karaman-Jurukovska, N.; Dong, K. K.; Damaghi, N.; Smiles, K. A.; Yarosh, D. B. Skin cells and tissue are capable of using L-ergothioneine as an integral component of their antioxidant defense system. Free Radic Biol Med 2009, 46, 1168-1176, doi:10.1016/j.freeradbiomed.2009.01.021.
30. Halliwell, B.; Cheah, I. K.; Tang, R. M. Y. Ergothioneine—a diet-derived antioxidant with therapeutic potential. FEBS Lett 2018, 592, 3357-3366, doi:10.1002/1873-3468.13123.
31. Cheah, I. K.; Feng, L.; Tang, R. M. Y.; Lim, K. H. C.; Halliwell, B. Ergothioneine levels in an elderly population decrease with age and incidence of cognitive decline; a risk factor for neurodegeneration? Biochem Biophys Res Commun 2016, 478, 162-167, doi:10.1016/j.bbrc.2016.07.074.
32. Behof, W. J.; Whitmore, C. A.; Haynes, J. R.; Rosenberg, A. J.; Tantawy, M. N.; Peterson, T. E.; Harrison, F. E.; Beelman, R. B.; Pham, W. A novel antioxidant ergothioneine PET radioligand for in vivo imaging applications. Scientific Reports 2021, 11, 18450, doi:10.1038/s41598-021-97925-w.
33. Tucker, L. B.; McCabe, J. T. Behavior of Male and Female C57BL/6J Mice Is More Consistent with Repeated Trials in the Elevated Zero Maze than in the Elevated Plus Maze. Front Behav Neurosci 2017, 11, 13, doi:10.3389/fnbeh.2017.00013.
34. Klunk, W. E.; Engler, H.; Nordberg, A.; Wang, Y.; Blomqvist, G.; Holt, D. P.; Bergstrom, M.; Savitcheva, I.; Huang, G. F.; Estrada, S.; et al. Imaging brain amyloid in Alzheimer's disease with Pittsburgh Compound-B. Ann Neurol 2004, 55, 306-319, doi:10.1002/ana.20009.
35. Eimer, W. A.; Vassar, R. Neuron loss in the 5XFAD mouse model of Alzheimer's disease correlates with intraneuronal Abeta42 accumulation and Caspase-3 activation. Mol Neurodegener 2013, 8, 2, doi: #N/A [pii] 10.1186/1750-1326-8-2.
36. Behof, W. J.; Whitmore, C. A.; Haynes, J. R.; Rosenberg, A. J.; Tantawy, M. N.; Peterson, T. E.; Harrison, F. E.; Beelman, R. B.; Wijesinghe, P.; Matsubara, J. A.; et al. Improved synthesis of an ergothioneine PET radioligand for imaging oxidative stress in Alzheimer's disease. FEBS Lett 2022, Online ahead of print.
37. Calsolaro, V.; Edison, P. Alterations in Glucose Metabolism in Alzheimer's Disease. Recent Pat Endocr Metab Immune Drug Discov 2016, 10, 31-39, doi:10.2174/1872214810666160615102809.
38. Nunomura, A.; Perry, G.; G., A.; Kirai, K.; Takeda, A.; Balraj, E. K.; Jones, P. K.; Ghanbari, H.; Wataya, T.; Shimohama, S.; et al. Oxidative damage is the earliest event in Alzheimer Disease. J Neuopathology & Experimental Neurology 2001, 60, 759-767.
39. Christen, Y. Oxidative stress and Alzheimer disease. Am J Clin Nutr 2000, 71, 621S-629S, doi:10.1093/ajcn/71.2.621s.
40. Mezzaroba, L.; Alfieri, D. F.; Colado Simao, A. N.; Vissoci Reiche, E. M. The role of zinc, copper, manganese and iron in neurodegenerative diseases. Neurotoxicology 2019, 74, 230-241, doi:10.1016/j.neuro.2019.07.007.
41. Langkammer, C.; Ropele, S.; Pirpamer, L.; Fazekas, F.; Schmidt, R. MRI for iron mapping in Alzheimer's disease. Neurodegener Dis 2014, 13, 189-191, doi:10.1159/000353756.
42. Tisdall, M. D.; Ohm, D. T.; Lobrovich, R.; Das, S. R.; Mizsei, G.; Prabhakaran, K.; Ittyerah, R.; Lim, S.; McMillan, C. T.; Wolk, D. A.; et al. Ex vivo MRI and histopathology detect novel iron-rich cortical inflammation in frontotemporal lobar degeneration with tau versus TDP-43 pathology. Neuroimage Clin 2021, 33, 102913, doi:10.1016/j.nic1.2021.102913.
43. Gong, N. J.; Dibb, R.; Bulk, M.; van der Weerd, L.; Liu, C. Imaging beta amyloid aggregation and iron accumulation in Alzheimer's disease using quantitative susceptibility mapping MRI. Neuroimage 2019, 191, 176-185, doi:10.1016/j.neuroimage.2019.02.019.
44. Markesbery, W. R. The role of oxidative stress in Alzheimer disease. Arch Neurol 1999, 56, 1449-1452, doi:10.1001/archneur.56.12.1449.
45. Filiz, G.; Price, K. A.; Caragounis, A.; Du, T.; Crouch, P. J.; White, A. R. The role of metals in modulating metalloprotease activity in the AD brain. Eur Biophys J 2008, 37, 315-321, doi:10.1007/s00249-007-0244-1.
46. Rao, K. S. J.; Rao, R. V.; Shanmugavelu, P.; Menon, R. B. Trace elements in Alzheimer's disease brain: a new hypothesis. Alz Rep. 1999, 2, 241-246.
47. Smith, M. A.; Peggy, L. R. H.; Lawrence, M. S.; Perry, G. Iron accumulation in Alzheimer disease is a source of redox-generated free radicals. PNAS 1997, 94, 9866-9868.
48. Campbell, A.; Smith, M. A.; Sayre, L. M.; Bondy, S. C.; Perry, G. Mechanisms by which metals promote events connected to neurodegenerative diseases. Brain Res Bull 2001, 55, 125-132, doi:10.1016/s0361-9230(01)00455-5.
49. Zatta, P.; Lucchini, R.; van Rensburg, S. J.; Taylor, A. The role of metals in neurodegenerative processes: aluminum, manganese, and zinc. Brain Res Bull 2003, 62, 15-28, doi:10.1016/s0361-9230(03)00182-5.
50. Zecca, L.; Youdim, M. B.; Riederer, P.; Connor, J. R.; Crichton, R. R. Iron, brain ageing and neurodegenerative disorders. Nat Rev Neurosci 2004, 5, 863-873, doi:10.1038/nrn1537.
51. Hegde, M. L.; Bharathi, P.; Suram, A.; Venugopal, C.; Jagannathan, R.; Poddar, P.; Srinivas, P.; Sambamurti, K.; Rao, K. J.; Scancar, J.; et al. Challenges associated with metal chelation therapy in Alzheimer's disease. J Alzheimers Dis 2009, 17, 457-468, doi:10.3233/JAD-2009-1068.
52. Huang, X.; Atwood, C. S.; Moir, R. D.; Hartshorn, M. A.; Vonsattel, J. P.; Tanzi, R. E.; Bush, A. I. Zinc-induced Alzheimer's Abeta1-40 aggregation is mediated by conformational factors. J Biol Chem 1997, 272, 26464-26470, doi:10.1074/jbc.272.42.26464.
53. Ibach, B.; Haen, E.; Marienhagen, J.; Hajak, G. Clioquinol treatment in familiar early onset of Alzheimer's disease: a case report. Pharmacopsychiatry 2005, 38, 178-179, doi:10.1055/s-2005-871241.
54. McLachlan, D. R.; Smith, W. L.; Kruck, T. P. Desferrioxamine and Alzheimer's disease: video home behavior assessment of clinical course and measures of brain aluminum. Ther Drug Monit 1993, 15, 602-607.
55. Engelhart, M. J.; Geerlings, M. I.; Ruitenberg, A.; van Swieten, J. C.; Hofman, A.; Witteman, J. C.; Breteler, M. M. Dietary intake of antioxidants and risk of Alzheimer disease. JAMA 2002, 287, 3223-3229, doi:10.1001/jama.287.24.3223.
56. Sano, M.; Ernesto, C.; Thomas, R. G.; Klauber, M. R.; Schafer, K.; Grundman, M.; Woodbury, P.; Growdon, J.; Cotman, C. W.; Pfeiffer, E.; et al. A controlled trial of selegiline, alpha-tocopherol, or both as treatment for Alzheimer's disease. The Alzheimer's Disease Cooperative Study. N Engl J Med 1997, 336, 1216-1222, doi:10.1056/NEJM199704243361704.
57. Lannfelt, L.; Blennow, K.; Zetterberg, H.; Batsman, S.; Ames, D.; Harrison, J.; Masters, C. L.; Targum, S.; Bush, A. I.; Murdoch, R.; et al. Safety, efficacy, and biomarker findings of PBT2 in targeting Abeta as a modifying therapy for Alzheimer's disease: a phase IIa, double-blind, randomised, placebo-controlled trial. Lancet Neurol 2008, 7, 779-786, doi:10.1016/51474-4422(08)70167-4.
58. Lee, H. P.; Zhu, X.; Casadesus, G.; Castellani, R. J.; Nunomura, A.; Smith, M. A.; Lee, H. G.; Perry, G. Antioxidant approaches for the treatment of Alzheimer's disease. Expert Rev Neurother 2010, 10, 1201-1208, doi:10.1586/ern.10.74.
59. Curzon, P.; Rustay, N. R.; Browman, K. E. Methods of behavior analysis in neuroscience, 2nd edition. Chapter 2: Cued and contextual fear conditioning for rodents 2009, CRC Press/Taylor & Francis.
60. McClure, R.; Ong, H.; Janve, V.; Barton, S.; Zhu, M.; Li, B.; Dawes, M.; Jerome, W. G.; Anderson, A.; Massion, P.; et al. Aerosol Delivery of Curcumin Reduced Amyloid-beta Deposition and Improved Cognitive Performance in a Transgenic Model of Alzheimer's Disease. J Alzheimers Dis 2017, 55, 797-811, doi:10.3233/JAD-160289.
61. Jones, C. P.; Boyd, K. L.; Wallace, J. M. Evaluation of Mice Undergoing Serial Oral Gavage While Awake or Anesthetized. J Am Assoc Lab Anim Sci 2016, 55, 805-810.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A pharmaceutical composition, comprising:
a pharmaceutical carrier and an effective dose of ergothioneine (ERGO) based positron emission tomography (PET), wherein the ERGO PET probe has one of the following structures: 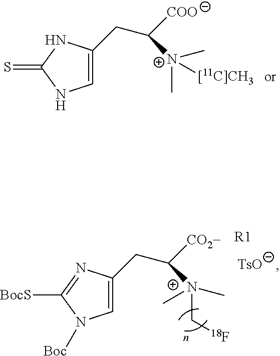
wherein n is 1, 2, or 3, wherein R1 is an alkyl group.

2. A precursor compound for ergothioneine (ERGO) based positron emission tomography (PET) probe having the following structure: 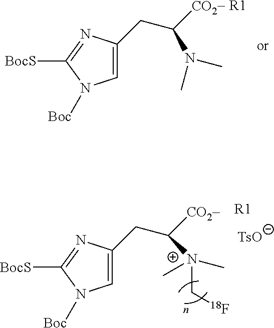
wherein n is 1, 2, or 3, wherein R1 is an alkyl group.

3. The precursor compound of claim 2, wherein R1 is a methyl group.

4. The precursor compound of claim 2, wherein R1 is a t-butyl group.

5. A method of imaging an inflammatory disease in a subject comprising:
administering to a subject a labeled probe in a detectably effective amount, wherein the labeled probe is ergothioneine (ERGO) based positron emission tomography (PET) probe having one of the following structures: 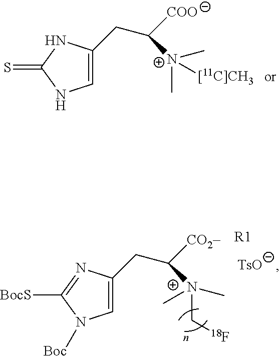
wherein n is 1, 2, or 3, wherein R1 is an alkyl group;
imaging at least a portion of the subject; and
detecting the labeled probe, wherein the location of the labeled probe corresponds to inflammation.

6. The method of claim 5, wherein the inflammation corresponds to the inflammatory disease.

7. The method of claim 5, wherein detection of the labeled probe in a location above a threshold is an indication of presence of the inflammatory disease at the location.

8. The method of claim 5, wherein dimensions of the location are monitored over time.

9. The method of claim 5, wherein the level of uptake of the labeled probe in a tissue corresponds to the level inflammation in the tissue.

10. The method of claim 5, wherein the detection of the labeled probe is performed in vitro using Positron Emission Tomography (PET), Computerized Tomography (CT), or a combination thereof.

11. The method of claim 5, wherein the inflammatory disease is selected from one of the following: Alzheimer's disease (AD), Huntington's disease, Amytrophic Lateral Sclerosis (ALS), or Parkinson's disease.
